SEQUENCE LISTING (14 sequences)

SEQ ID NO: 1 - DNA, 20 bp, Artificial Sequence
  Primer
agtgtgacgttgacatccgt

SEQ ID NO: 2 - DNA, 20 bp, Artificial Sequence
  Primer
gcagctcagtaacagtccgc

SEQ ID NO: 3 - DNA, 20 bp, Artificial Sequence
  Primer
ctttgtgcctctcgggatga

SEQ ID NO: 4 - DNA, 23 bp, Artificial Sequence
  Primer
aggttacttgagtctccggatct

SEQ ID NO: 5 - DNA, 20 bp, Artificial Sequence
  Primer
ctgcactcaagggactcctc

SEQ ID NO: 6 - DNA, 20 bp, Artificial Sequence
  Primer
gttgtcacctgcttgggttt

SEQ ID NO: 7 - DNA, 20 bp, Artificial Sequence
  Primer
ccgtgtttctcatggttgtg

SEQ ID NO: 8 - DNA, 20 bp, Artificial Sequence
  Primer
accagaaggagcagcactgt

SEQ ID NO: 9 - DNA, 20 bp, Artificial Sequence
  Primer
gagtttggggaccagtttga

SEQ ID NO: 10 - DNA, 20 bp, Artificial Sequence
  Primer
gcttcagggcttcttttcct

SEQ ID NO: 11 - DNA, 20 bp, Artificial Sequence
  Primer
tacccaaggtggagatcctg

SEQ ID NO: 12 - DNA, 20 bp, Artificial Sequence
  Primer
ctgggttccctgttctgtgt

SEQ ID NO: 13 - DNA, 21 bp, Artificial Sequence
  Primer
gcccaggaaaaccccaagatg

SEQ ID NO: 14 - DNA, 20 bp, Artificial Sequence
  Primer
ccgttagcccagccgttgac

(19) United States
(12) Patent Application Publication
(10) Pub. No.: US 2012/0164731 A1
(43) Pub. Date: Jun. 28, 2012

(54) METHOD OF INDUCING DIFFERENTIATION FROM PLURIPOTENT STEM CELLS TO SKELETAL MUSCLE PROGENITOR CELLS

(75) Inventors: Hidetoshi Sakurai, Kyoto (JP); Yasuko Sakaguchi, Kyoto (JP); Atsuko Sehara, Kyoto (JP)

(73) Assignee: KYOTO UNIVERSITY, Kyoto-shi, Kyoto (JP)

(21) Appl. No.: 13/382,470

(22) PCT Filed: Jul. 9, 2010
(86) PCT No.: PCT/JP2010/062023

§ 371 Date: Mar. 19, 2012

Related U.S. Application Data
(60) Provisional application No. 61/270,479, filed on Jul. 9, 2009.

(51) Int. Cl. C12N 5/0775

(52) U.S. Cl. 435/377

(57) ABSTRACT

Provided is a method of producing a skeletal muscle progenitor cell using a pluripotent stem cell, particularly an iPS cell, the method comprising the step 1) of culturing a pluripotent stem cell under serum-free conditions, and in the presence of Activin A, to allow the cell to differentiate into a PDGFRα-positive mesodermal cell, and the step 2) of culturing the mesodermal cell under serum-free conditions, and in the presence of a Wnt signal inducer, to allow the cell to differentiate into a skeletal muscle progenitor cell. Also provided are a cell population containing a skeletal muscle progenitor cell as obtained by the method, and a skeletal muscle regeneration promoting agent and therapeutic agent for muscular diseases such as muscular dystrophy, the promoting agent or agent comprising the skeletal muscle progenitor cell as an active ingredient.

### TECHNICAL FIELD

The present invention relates to a method of inducing differentiation from a pluripotent stem cell, particularly from an induced pluripotent stem cell, to a skeletal muscle progenitor cell, a reagent kit for use in the method, a skeletal muscle progenitor cell obtained by the method, and a treatment of myopathy using the skeletal muscle progenitor cell.

### BACKGROUND OF THE INVENTION

Although muscular diseases involve a wide variety of pathologic conditions, the symptoms manifested are for the most part muscular atrophy and associated weakness of muscles. Muscular atrophy can occur in two types: myogenic diseases (myopathy), in which muscles are disordered, and neurogenic diseases, in which nerves that control muscle motors are disordered. A representative myopathy is muscular dystrophy. Muscular dystrophy generically refers to hereditary muscular diseases characterized by gradual progression of muscular atrophy and weakness of muscles in repeated cycles of muscle fiber necrosis and regeneration, which are classified under a wide variety of disease types, involving different causal genes for respective disease types and different modes of mutations.

As a possible radical therapy for muscular dystrophy, stem cell transplantation has been proposed. Satellite cells between muscle fibers and basement membrane are muscular stem cells that had been known before. Later, it was found that stem cells capable of differentiating into muscles are present in marrow cells, which are relatively easy to collect, and which can be proliferated to some extent in vitro, so muscular stem cell transplantation therapy attracted attention. Because muscular dystrophy is a hereditary disease, however, the patient's own bone marrow cannot be used in the therapy, and even marrow cells are unable to proliferate infinitely.

Embryonic stem (ES) cells are capable of differentiating into almost all types of tissues, and can be proliferated nearly infinitely while maintaining the undifferentiated state. Darabi et al. recently succeeded in restoring some normal muscular function in a mouse model of muscular dystrophy by transferring the transcription factor Pax3, which promotes differentiation into myocytes, to mouse ES cells to induce muscle formation, sorting out skeletal muscle progenitor cells, and transplanting the sorted cells to the mouse model of muscular dystrophy [Darabi, R. et al., Nat. Med., 14: 134-143 (2008)]. Because of the unavoidable involvement of genetic manipulation for differentiation into skeletal muscle progenitor cells, however, this method cannot immediately be applied to human clinical practice. Furthermore, ethical issues with ES cells pose the problem of difficulty in procuring ES cells that match the patient.

In recent years, induced pluripotent stem cells (iPS cells) derived from somatic cells have been established, without embryo destruction, in mice, humans and other animals one after another [WO 2007/069666 A1; Takahashi, K. and Yamanaka, S., Cell, 126: 663-676 (2006); Okita, K. et al., Nature, 448: 313-317 (2007); Nakagawa, M. et al., Nat. Biotechnol., 26: 101-106 (2008); Takahashi, K. et al., Cell, 131: 861-872 (2007); Yu, J. et al., Science, 318: 1917-1920 (2007)], and are attracting attention as a source of graft cells that substitute for ES cells. Safer iPS cells not incorporating an extraneous gene have also been developed [Stadtfeld, M. et al., Science, 322: 945-949 (2008); Okita, K. et al., Science, 322: 949-953 (2008); Yu, J. et al., Science, 324: 797-801 (2009); Zhou, H. et al., Cell Stem Cell, 4: 381-384 (2009); Kim, D. et al., Cell Stem Cell, 4: 472-476 (2009)], and their application to regenerative medicine is becoming feasible. However, no report is available on successful induction of differentiation from an iPS cell to a skeletal muscle progenitor cell.

### SUMMARY OF THE INVENTION

An object of the present invention is to provide a method of inducing differentiation from pluripotent stem cells, including iPS cells, to skeletal muscle progenitor cells, without gene manipulation, by optimizing the culture conditions, and a differentiation induction reagent kit that comprises a medium ingredient to be added to the medium in the method. It is another object of the present invention to provide a therapeutic means for muscular diseases such as muscular dystrophy using skeletal muscle progenitor cells derived from pluripotent stem cells as obtained by the method.

The present inventors previously found that mouse ES cells can be induced to differentiate into skeletal muscle progenitor cells under serum-free conditions by culturing mouse ES cells in a medium containing bone morphogenetic protein 4 (BMP4), and then further culturing the cells in a medium containing lithium chloride (LiCl), and filed a patent application (Japanese Patent Application No. 2008-186348). Bearing in mind the fact that differentiation induction in ES cells is often effective also in iPS cells, the present inventors first attempted to apply the method to mouse iPS cells, but it was found, unexpectedly, that no cells survived in this method.

Hence, the present inventors conducted differentiation induction experiments with various growth factors in combination, and for the first time found that by culturing iPS cells in a medium containing Activin A (Medium A), and then further culturing in a medium containing a Wnt signal inducer such as LiCl (Medium B), the cells can be induced to differentiate into skeletal muscle progenitor cells in the absence of serum. Furthermore, the present inventors found that the differentiation induction efficiency could be increased by further adding BMP and/or insulin-like growth factor-1 (IGF-1) to Medium A, and/or further adding sonic hedgehog (Shh) and/or IGF-1 to Medium B.

When the skeletal muscle progenitor cells obtained were transplanted to muscular dystrophy model mice, many muscle fibers were observed, and an inflammation suppressing effect and muscle tissue repair effect were observed. Also observed was induction of differentiation into satellite cells and dystrophin-expressing cells. The present inventors conducted further investigations based on these findings, and have developed the present invention.

Accordingly, the present invention provides the following:

[1] A method of producing a skeletal muscle progenitor cell with the use of an iPS cell.
[2] The method according to [1] above, wherein the skeletal muscle progenitor cell is Myf5-positive and MyoD-positive.
[3] A method of producing a PDGFRα-positive mesodermal cell from a pluripotent stem cell, wherein the pluripotent stem cell is cultured under serum-free conditions and in the presence of Activin A.
[4] The method according to [3] above, wherein the PDGFRα-positive mesodermal cell is a primitive streak mesodermal cell.
[5] The method according to [3] or [4] above, wherein the pluripotent stem cell is cultured in the presence of further BMP and/or IGF-1.
[6] The method according to [5] above, wherein the BMP comprises at least one selected from among BMP2, BMP4 and BMP7.
[7] A method of producing a skeletal muscle progenitor cell from a PDGFRα-positive mesodermal cell, wherein the mesodermal cell is cultured under serum-free conditions and in the presence of a Wnt signal inducer.
[8] The method according to [7] above, wherein the skeletal muscle progenitor cell is Myf5-positive and MyoD-positive.
[9] The method according to [7] or [8] above, wherein the Wnt signal inducer comprises at least one selected from among LiCl, Wnt1, Wnt3a and Wnt7a.
[10] The method according to any one of [7] to [9] above, wherein the mesodermal cell is cultured in the presence of further Shh and/or IGF-1.
[11] The method according to any one of [7] to [10] above, wherein the PDGFRα-positive mesodermal cell is obtained by the method according to any one of [3] to [6] above.
[12] A method of producing a skeletal muscle progenitor cell from a pluripotent stem cell, wherein the following steps 1) and 2) are followed under serum-free conditions:
1) the step of culturing a pluripotent stem cell in the presence of Activin A,
2) the step of culturing the cell obtained in the foregoing step 1) in the presence of a Wnt signal inducer.
[13] The method according to [12] above, wherein the skeletal muscle progenitor cell is Myf5-positive and MyoD-positive.
[14] The method according to [12] or [13] above, wherein the Wnt signal inducer comprises at least one selected from among LiCl, Wnt1, Wnt3a and Wnt7a.
[15] The method according to any one of [12] to [14] above, wherein the pluripotent stem cell is cultured in the presence of further BMP and/or IGF-1 in the foregoing step 1).
[16] The method according to [15] above, wherein the BMP comprises at least one selected from among BMP2, BMP4 and BMP7.
[17] The method according to any one of [12] to [16] above, wherein the cell obtained in the foregoing step 1) is cultured in the presence of further Shh and/or IGF-1 in the foregoing step 2).
[18] The method according to [12] above, wherein the following steps 1) and 2) are followed:
1) the step of culturing a pluripotent stem cell in the presence of Activin A, BMP4 and IGF-1,
2) the step of culturing the cell obtained in the foregoing step 1) in the presence of LiCl, Shh and IGF-1.
[19] The method according to any one of [11] to [17] above, wherein the foregoing steps 1) and 2) are followed by the step 3):
3) the step of selecting a PDGFRα-positive cell from among the cells obtained in the foregoing step 2).
[20] The method according to any one of [3] to [6] and [11] to
[19] above, wherein the pluripotent stem cell is an iPS cell or ES cell.
[21] A reagent kit for inducing differentiation from a pluripotent stem cell to a PDGFRα-positive mesodermal cell, wherein the kit comprises Activin A, BMP4 and IGF-1.
[22] A reagent kit for inducing differentiation from a PDGFRα-positive mesodermal cell to a skeletal muscle progenitor cell, wherein the kit comprises LiCl, Shh and IGF-1.
[23] The kit according to [21] or [22] above, wherein the pluripotent stem cell is an iPS cell or ES cell.
[24] A cell population containing skeletal muscle progenitor cells produced by the method according to any one of [1], [2] and [7] to [20] above.
[25] The cell population according to [24] above, wherein the reprogramming genes are integrated in the genome.
[26] The cell population according to [25] above, wherein the reprogramming genes are 4 different genes consisting of Oct3/4, Sox2, Klf4 and c-Myc, or 3 different genes consisting of Oct3/4, Sox2 and Klf4.
[27] A skeletal muscle regeneration promoting agent comprising as an active ingredient a skeletal muscle progenitor cell contained in the cell population according to any one of [24] to [26] above.
[28] A satellite cell formation promoting agent comprising as an active ingredient a skeletal muscle progenitor cell contained in the cell population according to any one of [24] to [26] above.
[29] The agent according to [27] or [28] above, wherein the agent is a therapeutic agent for a muscular disease.
[30] The agent according to [29] above, wherein the muscular disease is muscular dystrophy.
[31] A method of skeletal muscle regeneration and/or satellite cell formation in a subject in need of skeletal muscle regeneration and/or satellite cell formation, comprising administering an effective amount of the cell population according to any one of [24] to [26] above to the subject.

According to the present invention, a pluripotent stem cell can be induced to differentiate into a skeletal muscle progenitor cell without gene manipulation by adding appropriately combined growth factors to the medium. The present invention also makes it possible to induce differentiation from an iPS cell to a skeletal muscle progenitor cell, enabling stable supply of skeletal muscle progenitor cells without being subject to ethical limitations as with ES cells. Furthermore, according to the present invention, pluripotent stem cells can be differentiated into skeletal muscle progenitor cells under serum-free conditions; therefore, lot-to-lot variation is small, skeletal muscle progenitor cells can be efficiently obtained using any cell clone, and applications to medical practice are possible.

Because skeletal muscle progenitor cells obtained by the present invention have a muscle inflammation suppressing effect and muscle tissue repair effect, they are expected to find applications for skeletal muscle regenerative medicine in muscular dystrophy and other muscular diseases.

### BRIEF DESCRIPTION OF THE DRAWINGS

FIG. 1A shows conditions for inducing differentiation from an iPS cell to a primitive streak mesodermal cell and a method of evaluation. FIG. 1B shows charts of a FACS analysis in which the effects of Activin A, IGF-1 and HGF on the induction of differentiation from an iPS cell to a primitive streak mesodermal cell were evaluated by the percentage of PDGFRα-positive cells (left), and a graph obtained by evaluating the effects by viable cell count (right). FIG. 1C shows charts of a FACS analysis in which the influence of seeded cell density on the induction of differentiation from an iPS cell to a primitive streak mesodermal cell was evaluated by the percentage of PDGFRα-positive cells.

FIG. 2A is a chart of a FACS analysis in which the influence of Activin A concentration on the induction of differentiation from an iPS cell to a primitive streak mesodermal cell was evaluated by the percentage of PDGFRα-positive cells. FIG. 2B is a chart of a FACS analysis in which the influence of BMP4 concentration on the induction of differentiation from an iPS cell to a primitive streak mesodermal cell was evaluated by the percentage of PDGFRα-positive cells. FIG. 2C is a graphic representation of the influence of Activin A and BMP4 concentrations on the induction of differentiation from an iPS cell to a primitive streak mesodermal cell, as evaluated by viable cell count.

FIG. 3A shows conditions for inducing differentiation from an iPS cell to a primitive streak mesodermal cell and conditions for inducing differentiation from a primitive streak mesodermal cell to a skeletal muscle progenitor cell, and a method of evaluation. FIG. 3B shows charts of a FACS analysis in which the effect of Sonic Hedgehog (Shh) on the induction of differentiation from a primitive streak mesodermal cell to a skeletal muscle progenitor cell was evaluated by the percentage of SM/C-2.6-positive cells (left), and a photograph of an RT-PCR in which the effect was evaluated by the expression of Myf5 (right). FIG. 3C shows charts of a FACS analysis in which the effect of IGF-1 on the induction of differentiation from a primitive streak mesodermal cell to a skeletal muscle progenitor cell was evaluated by the percentage of SM/C-2.6-positive cells (left), and a photograph of an RT-PCR in which the effect was evaluated by the expression of Myf5 (right).

FIG. 4A is a graphic representation showing results of a FACS analysis of a mesodermal cell population derived from iPS cells obtained by the differentiation induction method of the present invention with PDGFRα as a marker (left), and results of separation of the cell population into a PDGFRα-positive fraction and a negative fraction (right). FIG. 4B is a photographic representation of an RT-PCR showing results of an examination of the expression of various differentiation markers in RNAs extracted from the foregoing PDGFRα-positive fraction (panel 1) and negative fraction (panel 2). FIG. 4C is a graphic representation of a FACS analysis showing results of a comparison of the ratio of SM/C-2.6-positive cells and PDGFRα-positive cells. FIG. 4D is a photographic representation showing results of fluorescent immunostaining performed using antibodies against Pax7, DsRed, Laminin, and DAPI 4 weeks after PDGFRα-positive cells were transplanted to skeletal muscles of cardiotoxin-treated mice (upper row: Pax7 positive DsRed-positive cells inside the laminin, lower row: Pax7-negative DsRed-positive cells outside the laminin). FIG. 4E compares the numbers of DsRed-positive cells and Pax7-positive cells obtained after intramuscular injection (i.m.) and intravenous injection (i.v.) of PDGFRα-positive cells.

FIG. 5A is a photographic representation of results of an analysis of various tissues performed 4 weeks after intramuscular injection of skeletal muscle progenitor cells derived from iPS cells (PDGFRα-positive cells) into the tibialis anterior muscle (T.A.) of DMD-null mice. FIG. 5B is a photographic representation of results of fluorescent immunostaining of dystrophin in similar tissues. FIG. 5C is a photographic representation of results of fluorescent immunostaining of DsRed and dystrophin in similar tissues. FIG. 5D is a photographic representation of results of fluorescent immunostaining of DsRed and Pax7 in similar tissues.

FIG. 6A is a photographic representation showing the results of an analysis of various tissues performed 4 weeks after intramuscular injection of skeletal muscle progenitor cells derived from iPS cells (PDGFRα-positive cells) into the left tibialis anterior muscle (T.A.) of DMD-null mice. FIG. 6B is a photographic representation of results of fluorescent immunostaining of dystrophin in similar tissues. FIG. 6C is a photographic representation of results of fluorescent immunostaining of DsRed and SM/C-2.6 in similar tissues.

FIG. 7(a-d) are photographic representations showing the results of Myogenin immunostaining of mature skeletal muscle resulting from differentiation induction, in a test tube, of the PDGFRα-positive fraction and negative fraction obtained by the differentiation induction method of the present invention. FIG. 7(e) is a graph showing the ratio of the Myogenin-positive nucleus to the number of whole nuclei on the culture dish.

FIG. 8A is a graph showing the induction conditions and evaluation method when the differentiation induction method of the present invention is performed at different timing of LiCl addition. FIG. 8B is a graphic representation showing the results of FACS analysis of changes of the ratio of the PDGFRα-positive cells depending on the timing of LiCl addition.

### DETAILED DESCRIPTION OF THE INVENTION

The present invention provides a method of producing a skeletal muscle progenitor cell from a pluripotent stem cell, comprising the step 1) of differentiating a pluripotent stem cell into a PDGFRα-positive mesodermal cell, and the step 2) of differentiating the mesodermal cell into a skeletal muscle progenitor cell.

Accordingly, a first aspect of the present invention relates to a method of producing a PDGFRα-positive mesodermal cell from a pluripotent stem cell, comprising culturing a pluripotent stem cell under serum-free conditions, and in the presence of Activin A.

The pluripotent stem cell for use as the starting material may be any undifferentiated cell possessing a “self-renewal” that enables it to proliferate while retaining the undifferentiated state, and “pluripotency” that enables it to differentiate into all the three primary germ layers of the embryo. Examples include iPS cells, ES cells, embryonic germ (EG) cells, embryonic cancer (EC) cells and the like, with preference given to iPS cells or ES cells. The method of the present invention is applicable to any mammalian species for which any pluripotent stem cell line has been established or can be established. Examples of such mammals include humans, mice, rats, monkeys, dogs, pigs, bovines, cats, goat, sheep, rabbits, guinea pigs, hamsters and the like, with preference given to humans, mice, rats, monkeys, dogs and the like, more preferably humans or mice.

### (1) Preparation of Pluripotent Stem Cells

### (i) ES Cells

Pluripotent stem cells can be acquired by methods known per se. For example, available methods of preparing ES cells include, but are not limited to, methods in which a mammalian inner cell mass in the blastocyst stage is cultured [see, for example, Manipulating the Mouse Embryo: A Laboratory Manual, Second Edition, Cold Spring Harbor Laboratory Press (1994)] and methods in which an early embryo prepared by somatic cell nuclear transfer is cultured [Wilmut et al., Nature, 385, 810 (1997); Cibelli et al., Science, 280, 1256 (1998); Iritani et al., Protein, Nucleic Acid and Enzyme, 44, 892 (1999); Baguisi et al., Nature Biotechnology, 17, 456 (1999); Wakayama et al., Nature, 394, 369 (1998); Wakayama et al., Nature Genetics, 22, 127 (1999); Wakayama et al., Proc. Natl. Acad. Sci. USA, 96, 14984 (1999); Rideoutlll et al., Nature Genetics, 24, 109 (2000)].

### (ii) iPS Cells

An iPS cell can be prepared by transferring a nuclear reprogramming substance to a somatic cell.

### (a) Sources of Somatic Cells

Any cells other than germ cells of mammalian origin (e.g., mice, humans) can be used as starting material for the production of iPS cells. Examples include keratinizing epithelial cells (e.g., keratinized epidermal cells), mucosal epithelial cells (e.g., epithelial cells of the superficial layer of tongue), exocrine gland epithelial cells (e.g., mammary gland cells), hormone-secreting cells (e.g., adrenomedullary cells), cells for metabolism or storage (e.g., liver cells), intimal epithelial cells constituting interfaces (e.g., type I alveolar cells), intimal epithelial cells of the obturator canal (e.g., vascular endothelial cells), cells having cilia with transporting capability (e.g., airway epithelial cells), cells for extracellular matrix secretion (e.g., fibroblasts), constrictive cells (e.g., smooth muscle cells), cells of the blood and the immune system (e.g., T lymphocytes), sense-related cells (e.g., bacillary cells), autonomic nervous system neurons (e.g., cholinergic neurons), sustentacular cells of sensory organs and peripheral neurons (e.g., satellite cells), nerve cells and glia cells of the central nervous system (e.g., astroglia cells), pigment cells (e.g., retinal pigment epithelial cells), progenitor cells thereof (tissue progenitor cells) and the like. There is no limitation on the degree of cell differentiation; even undifferentiated progenitor cells (including somatic stem cells) and finally differentiated mature cells can be used alike as sources of somatic cells in the present invention. Examples of undifferentiated progenitor cells include tissue stem cells (somatic stem cells) such as nerve stem cells, hematopoietic stem cells, mesenchymal stem cells, and dental pulp stem cells.

The choice of mammal individual as a source of somatic cells is not particularly limited; however, when the iPS cells obtained are to be used for the treatment of nonhereditary myopathy in humans, it is preferable, from the viewpoint of prevention of graft rejection, that somatic cells are patient's own cells or collected from another person having the same or substantially the same HLA type as that of the patient. Meanwhile, if the iPS cell is to be used for the treatment of a hereditary muscular disease such as muscular dystrophy, it is preferable that the somatic cell be collected from a person, other than the patient, who has the normal gene, and whose HLA type is the same or substantially the same as the patient's. “Substantially the same HLA type” as used herein means that the HLA type of donor matches with that of patient to the extent that the transplanted cells, which have been obtained by inducing differentiation of iPS cells derived from the donor's somatic cells, can be engrafted when they are transplanted to the patient with use of immunosuppressor and the like. For example, it includes an HLA type wherein major HLAs (the three major loci of HLA-A, HLA-B and HLA-DR) are identical (hereinafter the same meaning shall apply) and the like. When the iPS cells obtained are not to be administered (transplanted) to a human, but used as, for example, a source of cells for screening for evaluating a patient's drug susceptibility or adverse reactions, it is likewise necessary to collect the somatic cells from the patient or another person with the same genetic polymorphism correlating with the drug susceptibility or adverse reactions.

### (b) Nuclear Reprogramming Substances

In the present invention, “a nuclear reprogramming substance” refers to any substance(s) capable of inducing an iPS cell from a somatic cell, which may be composed of any substance such as a proteinous factor or a nucleic acid that encodes the same (including forms incorporated in a vector), or a low-molecular compound. When the nuclear reprogramming substance is a proteinous factor or a nucleic acid that encodes the same, the following combinations, for example, are preferable (hereinafter, only the names for proteinous factors are shown).

(1) Oct3/4, Klf4, c-Myc
(2) Oct3/4, Klf4, c-Myc, Sox2 (Sox2 is replaceable with Sox1, Sox3, Sox15, Sox17 or Sox18; Klf4 is replaceable with Klf1, Klf2 or Klf5; c-Myc is replaceable with T58A (active mutant), N-Myc, or L-Myc)
(3) Oct3/4, Klf4, c-Myc, Sox2, Fbx15, Nanog, Eras, ECAT15-2, TclI, β-catenin (active mutant S33Y)
(4) Oct3/4, Klf4, c-Myc, Sox2, TERT, SV40 Large T antigen (hereinafter SV40LT)
(5) Oct3/4, Klf4, c-Myc, Sox2, TERT, HPV16 E6
(6) Oct3/4, Klf4, c-Myc, Sox2, TERT, HPV16 E7
(7) Oct3/4, Klf4, c-Myc, Sox2, TERT, HPV6 E6, HPV16 E7
(8) Oct3/4, Klf4, c-Myc, Sox2, TERT, Bmil
[For more information on the factors shown above, see WO 2007/069666 (for information on replacement of Sox2 with SoxlB and replacement of Klf4 with Klf1 or Klf5 in the combination (2) above, see Nature Biotechnology, 26, 101-106 (2008)); for the combination “Oct3/4, Klf4, c-Myc, Sox2”, see also Cell, 126, 663-676 (2006), Cell, 131, 861-872 (2007) and the like; for the combination “Oct3/4, Klf2 (or Klf5), c-Myc, Sox2”, see also Nat. Cell Biol., 11, 197-203 (2009); for the combination “Oct3/4, Klf4, c-Myc, Sox2, hTERT, SV40 LT”, see also Nature, 451, 141-146 (2008).]

### (9) Oct3/4, Klf4, Sox2 (see Nature Biotechnology, 26, 101-106 (2008))

### (10) Oct3/4, Sox2, Nanog, Lin28 (see Science, 318, 1917-1920 (2007))

(11) Oct3/4, Sox2, Nanog, Lin28, hTERT, SV40LT (see Stem Cells, 26, 1998-2005 (2008))
(12) Oct3/4, Klf4, c-Myc, Sox2, Nanog, Lin28 (see Cell Research (2008) 600-603)
(13) Oct3/4, Klf4, c-Myc, Sox2, SV40LT (see also Stem Cells, 26, 1998-2005 (2008)) (14) Oct3/4, Klf4 (see Nature 454:646-650 (2008), Cell Stem Cell, 2:525-528 (2008))
(15) Oct3/4, c-Myc (see Nature 454:646-650 (2008))

### (16) Oct3/4, Sox2 (see Nature, 451, 141-146 (2008), WO2008/118820)

### (17) Oct3/4, Sox2, Nanog (see WO2008/118820)

### (18) Oct3/4, Sox2, Lin28 (see WO2008/118820)

(19) Oct3/4, Sox2, c-Myc, Esrrb (Here, Essrrb can be substituted by Esrrg, see Nat. Cell Biol., 11, 197-203 (2009))
(20) Oct3/4, Sox2, Esrrb (see Nat. Cell Biol., 11, 197-203 (2009))

### (21) Oct3/4, Klf4, L-Myc

### (22) Oct3/4, Nanog

### (23) Oct3/4

(24) Oct3/4, Klf4, c-Myc, Sox2, Nanog, Lin28, SV40LT (see Science, 324: 797-801 (2009))

In (1)-(24) above, Oct3/4 may be replaced with another member of the Oct family, for example, Oct1A, Oct6 or the like. Sox2 (or Sox1, Sox3, Sox15, Sox17, Sox18) may be replaced with another member of the Sox family, for example, Sox7 or the like.

Furthermore, Lin28 may be replaced with another member of the Lin family, for example, Lin28b or the like.

Any combination that does not fall in (1) to (24) above but comprises all the constituents of any one of (1) to (24) above and further comprises an optionally chosen other substance can also be included in the scope of “nuclear reprogramming substances” in the present invention. Provided that the somatic cell to undergo nuclear reprogramming is endogenously expressing one or more of the constituents of any one of (1) to (24) above at a level sufficient to cause nuclear reprogramming, a combination of only the remaining constituents excluding the one or more constituents can also be included in the scope of “nuclear reprogramming substances” in the present invention.

Of these combinations, a combination of at least one, preferably two or more, more preferably three or more, selected from among Oct3/4, Sox2, Klf4, c-Myc, Nanog, Lin28 and SV40LT, is a preferable nuclear reprogramming substance.

Particularly, when the iPS cells obtained are to be used for therapeutic purposes, a combination of the three factors Oct3/4, Sox2 and Klf4 [combination (9) above] are preferably used. When the iPS cells obtained are not to be used for therapeutic purposes (e.g., used as an investigational tool for drug discovery screening and the like), the four factors Oct3/4, Sox2, Klf4 and c-Myc, or the five factors Oct3/4, Klf4, c-Myc, Sox2 and Lin28, or the six factors consisting of the five factors and Nanog [combination (12) above], and further, the seven factors consisting of the six factors and SV40 Large T [combination (24) above] are preferable.

Furthermore, the above-described combinations wherein c-Myc is replaced with L-Myc are also preferred as nuclear reprogramming substances.

Information on the mouse and human cDNA sequences of the aforementioned nuclear reprogramming substances is available with reference to the NCBI accession numbers mentioned in WO 2007/069666 (in the publication, Nanog is described as ECAT4. Mouse and human cDNA sequence information on Lin28, Lin28b, Esrrb, Esrrg and L-Myc can be acquired by referring to the following NCBI accession numbers, respectively); those skilled in the art are easily able to isolate these cDNAs.

| Name of gene | Mouse | Human |
|---|---|---|
| Lin28 | NM_145833 | NM_024674 |
| Lin28b | NM_001031772 | NM_001004317 |
| Esrrb | NM_011934 | NM_004452 |
| Esrrg | NM_011935 | NM_001438 |
| L-Myc | NM_008506 | NM_001033081 |

A proteinous factor for use as a nuclear reprogramming substance can be prepared by inserting the cDNA obtained into an appropriate expression vector, introducing the vector into a host cell, and recovering the recombinant proteinous factor from the cultured cell or its conditioned medium. Meanwhile, when the nuclear reprogramming substance used is a nucleic acid that encodes a proteinous factor, the cDNA obtained is inserted into a viral vector, plasmid vector, episomal vector etc. to construct an expression vector, and the vector is subjected to the step of nuclear reprogramming.

### (c) Method of Transferring a Nuclear Reprogramming Substance to a Somatic Cell

Transfer of a nuclear reprogramming substance to a somatic cell can be achieved using a method known per se for protein transfer into a cell, provided that the substance is a proteinous factor. An advantage of the method of the present invention for producing a skeletal muscle progenitor cell resides in the possibility of inducing differentiation into a skeletal muscle progenitor cell without gene manipulation. Likewise, in view of human clinical applications, it is preferable that the starting material iPS cell be also prepared without gene manipulation.

Such methods include, for example, the method using a protein transfer reagent, the method using a protein transfer domain (PTD)—or cell penetrating peptide (CPP)—fusion protein, the microinjection method and the like. Protein transfer reagents are commercially available, including those based on a cationic lipid, such as BioPOTER Protein Delivery Reagent (Gene Therapy Systems), Pro-Ject™ Protein Transfection Reagent (PIERCE) and ProVectin (IMGENEX); those based on a lipid, such as Profect-1 (Targeting Systems); those based on a membrane-permeable peptide, such as Penetrain Peptide (Q biogene) and Chariot Kit (Active Motif), GenomONE (ISHIHARA SANGYO KAISHA, LTD.) utilizing HVJ envelope (inactivated hemagglutinating virus of Japan) and the like. The transfer can be achieved per the protocols attached to these reagents, a common procedure being as described below. Nuclear reprogramming substance(s) is(are) diluted in an appropriate solvent (e.g., a buffer solution such as PBS or HEPES), a transfer reagent is added, the mixture is incubated at room temperature for about 5 to 15 minutes to form a complex, this complex is added to cells after exchanging the medium with a serum-free medium, and the cells are incubated at 37° C. for one to several hours. Thereafter, the medium is removed and replaced with a serum-containing medium.

Developed PTDs include those using transcellular domains of proteins such as drosophila-derived AntP, HIV-derived TAT (Frankel, A. et al, Cell 55, 1189-93 (1988) or Green, M. & Loewenstein, P. M. Cell 55, 1179-88 (1988)), Penetratin (Derossi, D. et al, J. Biol. Chem. 269, 10444-50 (1994)), Buforin II (Park, C. B. et al. Proc. Natl. Acad. Sci. USA 97, 8245-50 (2000)), Transportan (Pooga, M. et al. FASEB J. 12, 67-77 (1998)), MAP (model amphipathic peptide) (Oehlke, J. et al. Biochim. Biophys. Acta. 1414, 127-39 (1998)), K-FGF (Lin, Y. Z. et al. J. Biol. Chem. 270, 14255-14258 (1995)), Ku70 (Sawada, M. et al. Nature Cell Biol. 5, 352-7 (2003)), Prion (Lundberg, P. et al. Biochem. Biophys. Res. Commun. 299, 85-90 (2002)), pVEC (Elmquist, A. et al. Exp. Cell Res. 269, 237-44 (2001)), Pep-1 (Morris, M. C. et al. Nature Biotechnol. 19, 1173-6 (2001)), Pep-7 (Gao, C. et al. Bioorg. Med. Chem. 10, 4057-65 (2002)), SynB1 (Rousselle, C. et al. Mol. Pharmacol. 57, 679-86 (2000)), HN-I (Hong, F. D. & Clayman, G L. Cancer Res. 60, 6551-6 (2000)), and HSV-derived VP22. CPPs derived from the PTDs include polyarginines such as 11R (Cell Stem Cell, 4,381-384 (2009)) and 9R (Cell Stem Cell, 4, 472-476 (2009)).

A fused protein expression vector incorporating cDNA of a nuclear reprogramming substance and PTD or CPP sequence is prepared, and recombination expression is performed using the vector. The fused protein is recovered and used for transfer. Transfer can be performed in the same manner as above except that a protin transfer reagent is not added.

Microinjection, a method of placing a protein solution in a glass needle having a tip diameter of about 1 μm, and injecting the solution into a cell, ensures the transfer of the protein into the cell.

However, taking into account the efficiency of establishment of iPS cells, nuclear reprogramming substance may also be used preferably in the form of a nucleic acid that encodes a proteinous factor, rather than the factor as it is. The nucleic acid may be a DNA or an RNA, or a DNA/RNA chimera, and may be double-stranded or single-stranded. Preferably, the nucleic acid is a double-stranded DNA, particularly a cDNA.

A cDNA of a nuclear reprogramming substance is inserted into an appropriate expression vector comprising a promoter capable of functioning in a host somatic cell. Useful expression vectors include, for example, viral vectors such as retrovirus, lentivirus, adenovirus, adeno-associated virus, herpesvirus and Sendai virus, plasmids for the expression in animal cells (e.g., pA1-11, pXT1, pRc/CMV, pRc/RSV, pcDNAI/Neo) and the like.

A vector for this purpose can be chosen as appropriate according to the intended use of the iPS cell to be obtained. Useful vectors include adenovirus vector, plasmid vector, adeno-associated virus vector, retrovirus vector, lentivirus vector, Sendai virus vector, episomal vector and the like.

Examples of promoters used in expression vectors include the EFla promoter, the CAG promoter, the SRα promoter, the SV40 promoter, the LTR promoter, the CMV (cytomegalovirus) promoter, the RSV (Rous sarcoma virus) promoter, the MoMuLV (Moloney mouse leukemia virus) LTR, the HSV-TK (herpes simplex virus thymidine kinase) promoter and the like, with preference given to the EF1α promoter, the CAG promoter, the MoMuLV LTR, the CMV promoter, the SRα promoter and the like.

The expression vector may contain as desired, in addition to a promoter, an enhancer, a polyadenylation signal, a selectable marker gene, a SV40 replication origin and the like. Examples of selectable marker genes include the dihydrofolate reductase gene, the neomycin resistant gene, the puromycin resistant gene and the like.

An expression vector harboring a nucleic acid as a nuclear reprogramming substance can be introduced into a cell by a technique known per se according to the choice of the vector. In the case of a viral vector, for example, a plasmid containing the nucleic acid is introduced into an appropriate packaging cell (e.g., Plat-E cells) or a complementary cell line (e.g., 293-cells), the viral vector produced in the culture supernatant is recovered, and the vector is infected to the cell by a method suitable for the viral vector. For example, specific means using a retroviral vector are disclosed in WO2007/69666, Cell, 126, 663-676 (2006) and Cell, 131, 861-872 (2007). Specific means using a lentivirus vector is disclosed in Science, 318, 1917-1920 (2007). When iPS cells are utilized as cell sources for regenerative medicine, an expression (reactivation) of a reprogramming gene potentially increases the risk of carcinogenesis in skeletal muscle tissues regenerated from skeletal muscle progenitor cells derived from iPS cells; therefore, a nucleic acid encoding a nuclear reprogramming substance is preferably expressed transiently, without being integrated into the chromosome of the cells.

From this viewpoint, use of an adenoviral vector, whose integration into chromosome is rare, is preferred. Specific means using an adenoviral vector is disclosed in Science, 322, 945-949 (2008). Because an adeno-associated viral vector is also low in the frequency of integration into chromosome, and is lower than adenoviral vectors in terms of cytotoxicity and inflammation-inducibility, it can be mentioned as another preferred vector. Because Sendai viral vector is capable of being stably present outside the chromosome, and can be degraded and removed using an siRNA as required, it is preferably utilized as well. Regarding a Sendai viral vector, one described in J. Biol. Chem., 282, 27383-27391 (2007) and JP-3602058 B can be used.

When a retroviral vector or a lentiviral vector is used, even if silencing of the transgene has occurred, it possibly becomes reactivated; therefore, for example, a method can be used preferably wherein a nucleic acid encoding a nuclear reprogramming substance is cut out using the Cre-loxP system, when becoming unnecessary. That is, with loxP sequences arranged on both ends of the nucleic acid in advance, iPS cells are induced, thereafter the Cre recombinase is allowed to act on the cells using a plasmid vector or adenoviral vector, and the region sandwiched by the loxP sequences can be cut out. Because the enhancer-promoter sequence of the LTR U3 region possibly upregulates a host gene in the vicinity thereof by insertion mutation, it is more preferable to avoid the expression regulation of the endogenous gene by the LTR outside of the loxP sequence remaining in the genome without being cut out, using a 3′-self-inactivating (SIN) LTR prepared by deleting the sequence, or substituting the sequence with a polyadenylation sequence such as of SV40. Specific means using the Cre-loxP system and SIN LTR is disclosed in Chang et al., Stem Cells, 27: 1042-1049 (2009).

Meanwhile, being a non-viral vector, a plasmid vector can be transferred into a cell using the lipofection method, liposome method, electroporation method, calcium phosphate co-precipitation method, DEAE dextran method, microinjection method, gene gun method and the like. Specific means using a plasmid as a vector are described in, for example, Science, 322, 949-953 (2008) and the like.

When a plasmid vector, an adenovirus vector and the like are used, the transfection can be performed once or more optionally chosen times (e.g., once to 10 times, once to 5 times or the like). When two or more kinds of expression vectors are introduced into a somatic cell, it is preferable that these all kinds of expression vectors be concurrently introduced into a somatic cell; however, even in this case, the transfection can be performed once or more optionally chosen times (e.g., once to 10 times, once to 5 times or the like), preferably the transfection can be repeatedly performed twice or more (e.g., 3 times or 4 times).

Also when an adenovirus or a plasmid is used, the transgene can get integrated into chromosome; therefore, it is eventually necessary to confirm the absence of insertion of the gene into chromosome by Southern blotting or PCR. For this reason, like the aforementioned Cre-loxP system, it can be advantageous to use a means wherein the transgene is integrated into chromosome, thereafter the gene is removed. In another preferred mode of embodiment, a method can be used wherein the transgene is integrated into chromosome using a transposon, thereafter a transposase is allowed to act on the cell using a plasmid vector or adenoviral vector so as to completely eliminate the transgene from the chromosome. As examples of preferable transposons, piggyBac, a transposon derived from a lepidopterous insect, and the like can be mentioned. Specific means using the piggyBac transposon is disclosed in Kaji, K. et al., Nature, 458: 771-775 (2009), Woltjen et al., Nature, 458: 766-770 (2009).

Another preferable non-integration type vector is an episomal vector, which is autonomously replicable outside chromosome. Specific means using an episomal vector is disclosed in Science, 324, 797-801 (2009).

When the nuclear reprogramming substance is a low-molecular compound, introduction thereof into a somatic cell can be achieved by dissolving the substance at an appropriate concentration in an aqueous or non-aqueous solvent, adding the solution to a medium suitable for cultivation of somatic cells isolated from human or mouse [e.g., minimal essential medium (MEM) comprising about 5 to 20% fetal bovine serum, Dulbecco's modified Eagle medium (DMEM), RPMI1640 medium, 199 medium, F12 medium, and the like] so that the nuclear reprogramming substance concentration will fall in a range that is sufficient to cause nuclear reprogramming in somatic cells and does not cause cytotoxicity, and culturing the cells for a given period. The nuclear reprogramming substance concentration varies depending on the kind of nuclear reprogramming substance used, and is chosen as appropriate over the range of about 0.1 nM to about 100 nM. Duration of contact is not particularly limited, as far as it is sufficient to cause nuclear reprogramming of the cells; usually, the nuclear reprogramming substance may be allowed to be co-present in the medium until a positive colony emerges.

### (d) iPS Cell Establishment Efficiency Improvers

In recent years, various substances that improve the efficiency of establishment of iPS cells, which has traditionally been low, have been proposed one after another. When brought into contact with a somatic cell together with the aforementioned nuclear reprogramming substances, these establishment efficiency improvers are expected to further raise the efficiency of establishment of iPS cells.

Examples of iPS cell establishment efficiency improvers include, but are not limited to, histone deacetylase (HDAC) inhibitors [e.g., valproic acid (VPA) (Nat. Biotechnol., 26(7): 795-797 (2008)], low-molecular inhibitors such as trichostatin A, sodium butyrate, MC 1293, and M344, nucleic acid-based expression inhibitors such as siRNAs and shRNAs against HDAC (e.g., HDAC1 siRNA Smartpool® (Millipore), HuSH 29mer shRNA Constructs against HDAC1 (OriGene) and the like), and the like], DNA methyltransferase inhibitors (e.g., 5′-azacytidine) [Nat. Biotechnol., 26(7): 795-797 (2008)], G9a histone methyltransferase inhibitors [e.g., low-molecular inhibitors such as BIX-01294 (Cell Stem Cell, 2: 525-528 (2008)), nucleic acid-based expression inhibitors such as siRNAs and shRNAs against G9a (e.g., G9a siRNA (human) (Santa Cruz Biotechnology) and the like) and the like], L-channel calcium agonists (e.g., Bayk8644) [Cell Stem Cell, 3, 568-574 (2008)], p53 inhibitors [e.g., siRNA and shRNA against p53 (Cell Stem Cell, 3, 475-479 (2008)), UTF1 [Cell Stem Cell, 3, 475-479 (2008)], Wnt Signaling (e.g., soluble Wnt3a) [Cell Stem Cell, 3, 132-135 (2008)], 2i/LIF [2i is an inhibitor of mitogen-activated protein kinase signaling and glycogen synthase kinase-3, PloS Biology, 6(10), 2237-2247 (2008)] and the like. As mentioned above, the nucleic acid-based expression inhibitors may be in the form of expression vectors harboring a DNA that encodes an siRNA or shRNA.

Among the constituents of the aforementioned nuclear reprogramming substances, SV40 large T and the like, for example, can also be included in the scope of iPS cell establishment efficiency improvers because they are deemed not essential, but auxiliary, factors for somatic cell nuclear reprogramming. In the situation of the mechanisms for nuclear programming remaining unclear, the auxiliary factors, which are not essential for nuclear reprogramming, may be conveniently considered as nuclear reprogramming substances or iPS cell establishment efficiency improvers. Hence, because the somatic cell nuclear reprogramming process is understood as an overall event resulting from contact of nuclear reprogramming substance(s) and iPS cell establishment efficiency improver(s) with a somatic cell, it seems unnecessary for those skilled in the art to always distinguish between the nuclear reprogramming substance and the iPS cell establishment efficiency improver.

Contact of an iPS cell establishment efficiency improver with a somatic cell can be achieved as described above for each of three cases: (a) the improver is a proteinous factor, (b) the improver is a nucleic acid that encodes the proteinous factor, and (c) the improver is a low-molecular compound.

An iPS cell establishment efficiency improver may be brought into contact with a somatic cell simultaneously with a nuclear reprogramming substance, or either one may be contacted in advance, as far as the efficiency of establishment of iPS cells from the somatic cell is significantly improved, compared with the absence of the improver. In an embodiment, for example, when the nuclear reprogramming substance is a nucleic acid that encodes a proteinous factor and the iPS cell establishment efficiency improver is a chemical inhibitor, the iPS cell establishment efficiency improver can be added to the medium after the cell is cultured for a given length of time after the gene transfer treatment, because the nuclear reprogramming substance involves a given length of time lag from the gene transfer treatment to the mass-expression of the proteinous factor, whereas the iPS cell establishment efficiency improver is capable of rapidly acting on the cell. In another embodiment, when a nuclear reprogramming substance and an iPS cell establishment efficiency improver are both used in the form of a viral or plasmid vector, for example, both may be simultaneously introduced into the cell.

Somatic cells separated from a mammal can be pre-cultured using a medium known per se suitable for the cultivation thereof, depending on the kind of the cells. Examples of such media include, but are not limited to, a minimal essential medium (MEM) containing about 5 to 20% fetal calf serum, Dulbecco's modified Eagle medium (DMEM), RPMI1640 medium, 199 medium, F12 medium, and the like. When using, for example, a transfection reagent such as a cationic liposome in contacting the cell with nuclear reprogramming substance(s) and iPS cell establishment efficiency improver(s), it is sometimes preferable that the medium be previously replaced with a serum-free medium to prevent a reduction in the transfer efficiency. After the nuclear reprogramming substance(s) (and iPS cell establishment efficiency improver(s)) is(are) brought into contact with the cell, the cell can be cultured under conditions suitable for the cultivation of, for example, ES cells. In the case of mouse cells, the cultivation is carried out with the addition of Leukemia Inhibitory Factor (LIF) as a differentiation suppressor to an ordinary medium. Meanwhile, in the case of human cells, it is desirable that basic fibroblast growth factor (bFGF) and/or stem cell factor (SCF) be added in place of LIF. Usually, the cells are cultured in the co-presence of mouse embryo-derived fibroblasts (MEFs) treated with radiation or an antibiotic to terminate the cell division thereof, as feeder cells. Usually, STO cells and the like are commonly used as MEFs, but for inducing iPS cells, SNL cells [McMahon, A. P. & Bradley, A. Cell 62, 1073-1085 (1990)] and the like are commonly used. Co-culture with feeder cells may be started before contact of the nuclear reprogramming substance, at the time of the contact, or after the contact (e.g., 1-10 days later).

A candidate colony of iPS cells can be selected by a method with drug resistance and reporter activity as indicators, and also by a method based on visual examination of morphology. As an example of the former, a colony positive for drug resistance and/or reporter activity is selected using a recombinant somatic cell wherein a drug resistance gene and/or a reporter gene is targeted to the locus of a gene highly expressed specifically in pluripotent cells (e.g., Fbx15, Nanog, Oct3/4 and the like, preferably Nanog or Oct3/4). Examples of such recombinant somatic cells include MEFs from a mouse having the βgeo (which encodes a fusion protein of β-galactosidase and neomycin phosphotransferase) gene knocked-in to the Fbx15 locus [Takahashi & Yamanaka, Cell, 126, 663-676 (2006)], MEFs from a transgenic mouse having the green fluorescent protein (GFP) gene and the puromycin resistance gene integrated in the Nanog locus [Okita et al., Nature, 448, 313-317 (2007)] and the like. Meanwhile, examples of the method of selecting candidate colonies based on visual examination of morphology include the method described by Takahashi et al. in Cell, 131, 861-872 (2007). Although the method using reporter cells is convenient and efficient, it is desirable from the viewpoint of safety that colonies be selected by visual examination when iPS cells are prepared for the purpose of human treatment. When the three factors Oct3/4, Klf4 and Sox2 are used as nuclear reprogramming substances, the number of clones established decreases but the resulting colonies are mostly of iPS cells of high quality comparable to ES cells, so that iPS cells can efficiently be established even without using reporter cells.

The identity of the cells of a selected colony as iPS cells can be confirmed by positive responses to a Nanog (or Oct3/4) reporter (puromycin resistance, GFP positivity and the like) as well as by the formation of a visible ES cell-like colony, as described above. However, to ensure higher accuracy, it is possible to perform tests such as analyzing the expression of various ES-cell-specific genes and transplanting the cells selected to a mouse and confirming the formation of teratomas.

(iii) Naive Human ES and iPS Cells

Conventional human ES cells derived from blastocyst-stage embryos have very different biological (morphological, molecular and functional) properties from mouse ES cells. Mouse pluripotent stem cells can exit in two functionally distinct states, LIF-dependent ES cells and bFGF-dependent epiblast stem cells (EpiSCs). Molecular analyses suggest that the pluripotent state of human ES cells is similar to that of mouse EPiSCs rather than that of mouse ES cells. Recently, human ES and iPS cells in a mouse ES cell-like pluripotent state (also referred to as naive human ES and iPS cells) have been established by ectopic induction of Oct3/4, Sox2, Klf4, c-Myc and Nanog in the presence of LIF (see Cell Stem Cells, 6: 535-546, 2010), or ectopic induction of Oct3/4, Klf4 and Klf2 combined with LIF and inhibitors of GSK3P and ERK1/2 pathway (see Proc. Natl. Acad. Sci. USA, online publication doi/10.1073/pnas.1004584107). These naive human ES and iPS cells may be prefarable starting materials for the present invention due to their pluripotent more immature compared to that of conventional human ES and iPS cells.

(2) Induction of Differentiation from Pluripotent Stem Cells to PDGFRα-Positive Mesodermal Cells

Basal media for differentiation induction include, but are not limited to, serum-free minimal essential medium (MEM), Dulbecco's modified Eagle medium (DMEM), RPMI1640 medium, 199 medium, F12 medium, mixtures thereof, and media prepared by supplementing any one of the aforementioned media with appropriate concentrations of publicly known medium additives in common use [e.g., serum albumin, 2-mercaptoethanol, insulin, transferrin, sodium selenite, ethanolamine, antibiotics (e.g., penicillin, streptomycin) and the like] [e.g., S-Clone medium (e.g., SF-03, Sanko Junyaku)] and the like.

The differentiation induction medium of the present invention for inducing differentiation from a pluripotent stem cell to a PDGFRα-positive mesodermal cells (medium A) contains Activin A as an essential additive in the basal medium. Activin A increases cell survival dose-dependently in the induction of differentiation from a pluripotent stem cell to a PDGFRα-positive mesodermal cell. The Activin A concentration is, for example, about 1 ng/ml or more, preferably about 3 ng/ml or more, more preferably about 5 ng/ml or more, and is, for example, about 20 ng/ml or less, preferably about 15 ng/ml or less, more preferably 10 ng/ml or less.

The medium A preferably further contains BMP and/or IGF-1. BMP remarkably increases the induction efficiency for PDGFRα-positive cells when present in a range of effective concentrations. Examples of BMP include BMP2, BMP4, BMP7 and the like. One kind of BMP may be used alone, and 2 kinds or more may be used in combination. Preferably, 1 kind of BMP4 is used alone, or in combination with another or more kinds of BMP. The BMP concentration, as calculated for all kinds of BMP together, is, for example, about 5 ng/ml or more, preferably about 7.5 ng/ml or more, more preferably about 10 ng/ml or more, and is, for example, about 30 ng/ml or less, preferably about 20 ng/ml or less, more preferably about 15 ng/ml or less.

Meanwhile, even in the presence of Activin A alone or both Activin A and BMP, cell viability decreases if IGF-1 is absent; therefore, IGF-1 is thought to have a major influence at least on cell survival in this differentiation induction process. The IGF-1 concentration is, for example, about 1 ng/ml or more, preferably about 5 ng/ml or more, more preferably about 10 ng/ml or more, and is, for example, about 30 ng/ml or less, preferably about 20 ng/ml or less, more preferably about 15 ng/ml or less.

In a particularly preferred embodiment, the medium A contains Activin A, BMP4 and IGF-1 in addition to the basal medium. Appropriate concentrations of these ingredients can be chosen over the range of about 3 to 15 ng/ml, preferably about 5 to 10 ng/ml, for Activin A, about 7.5 to 20 ng/ml, preferably about 10 to 15 ng/ml, for BMP4, and about 5 to 20 ng/ml, preferably about 10 to 15 ng/ml, for IGF-1.

In this cultivation, pluripotent stem cells are seeded to a culture vessel known per se (e.g., gelatin- or collagen-coated 10 cm cell culture dish) to obtain a cell density of, for example, about 3 to 10×10⁴ cells/ml, preferably about 4% to 8×10⁴ cells/mL (about 3 to 10×10⁵ cells/10 cm dish, preferably about 4 to 8×10⁵ cells/10 cm dish), and cultured in an incubator under atmospheric conditions of 5% CO₂/95% air at about 30 to 40° C., preferably about 37° C., for about 2 to 7 days, preferably about 3 to 4 days. The fact of differentiation into PDGFRα-positive mesodermal cells can be confirmed by, for example, analyzing the phenotype of a cell surface antigen using an antibody against PDGFRα and a cell sorter. As required, the expression of another cell surface antigen or transcription factor can also be examined. Examples of the other surface antigen include Flk1 and VEGFR2. Examples of the transcription factor include brachyury(T) and Mix11. The pluripotent stem cells first differentiate into primitive streak mesodermal cells, which are the most immature type of PDGFRα/Flk1 double-positive cells, and then into PDGFRα-positive/Flk1-negative paraxial mesodermal cells, which are destined to differentiate into muscle cells. Meanwhile, lateral plate mesodermal cells, which are destined to differentiate into hemocytes and myocardial cells, exhibit the PDGFRα-negative/Flk1-positive phenotype. Because the PDGFRα-positive cell fraction obtained by this step of differentiation induction expresses Flk1 and brachyury(T), the majority of the cells contained in the fraction are thought to be in the stage of differentiation into primitive streak mesodermal cells.

As stated above, in a preferred embodiment of a first aspect of the present invention, Activin A, BMP4 and IGF-1 are contained in the medium A. Accordingly, the present invention also provides a reagent kit for induction of differentiation from a pluripotent stem cell to a PDGFRα-positive mesodermal cell, the kit comprising Activin A, BMP4 and IGF-1. These ingredients may be supplied in a state dissolved in water or an appropriate buffer solution, and may also be supplied as a lyophilized powder which may be used after being freshly dissolved in an appropriate solvent. These ingredients may be supplied as individual reagents in respective kits, and, as far as they do not adversely affect each other, they can be supplied as a single mixed reagent of 2 kinds or more.

(3) Induction of Differentiation from PDGFRα-Positive Mesodermal Cells to Skeletal Muscle Progenitor Cells

By culturing the thus-obtained PDGFRα-positive mesodermal cells under serum-free conditions and in the presence of a Wnt signal inducer, it is possible to induce differentiation into skeletal muscle progenitor cells. Accordingly, a second aspect of the present invention relates to a method of producing a skeletal muscle progenitor cell from a PDGFRα-positive mesodermal cell.

The PDGFRα-positive mesodermal cells to be treated in this step of differentiation induction (step 2) are not limited to those obtained in the step 1) described in detail in (2) above, and may be prepared by any method. For example, PDGFRα-positive mesodermal cells obtained by culturing ES cells in a BMP4-containing medium can be used. However, preferably, the PDGFRα-positive mesodermal cells to be treated in the step 2) are PDGFRα-positive mesodermal cells of pluripotent stem cell derivation, preferably of iPS cell or ES cell derivation, prepared in the step 1).

As the basal medium for differentiation induction in the step 2), a serum-free medium of the same composition as the foregoing step 1) is likewise preferably used.

The differentiation induction medium of the present invention for induction of differentiation from PDGFRα-positive mesodermal cells to skeletal muscle progenitor cells (medium B) contains at least 1 kind of Wnt signal inducer as an essential additive in the basal medium. Examples of the Wnt signal inducer include LiCl, Wnt1, Wnt3a, Wnt7a and the like. The Wnt signals mediated by Wnt1, Wnt3a, Wnt7a and the like positively control the expression of Myf5 and MyoD, which are transcription factors involved in muscle genesis, and LiCl is known as a classical activator of Wnt signals. Preferably, LiCl is used as a Wnt signal inducer. The concentration of Wnt signal inducer is, for example, about 1 mM or more, preferably about 3 mM or more, more preferably about 5 mM or more. Also, the concentration of Wnt signal inducer is, for example, about 20 mM or less, preferably about 15 mM or less, more preferably 10 mM or less.

The medium B preferably further contains Shh and/or IGF-1. Shh and IGF-1 remarkably increase skeletal muscle progenitor cell induction efficiency when present in ranges of effective concentrations. The concentration of Shh is, for example, about 5 ng/ml or more, preferably about 10 ng/ml or more, more preferably about 15 ng/ml or more. Also, the concentration of Shh is, for example, about 50 ng/ml or less, preferably about 30 ng/ml or less, more preferably about 25 ng/ml or less. Meanwhile, the concentration of IGF-1 is, for example, about 1 ng/ml or more, preferably about 5 ng/ml or more. Also, the concentration of IGF-1 is, for example, about 40 ng/ml or less, preferably about 20 ng/ml or less.

In a particularly preferred embodiment, the medium B contains LiCl, Shh and IGF-1 in addition to the basal medium. The concentrations of these ingredients can be chosen as appropriate over the range of about 3 to 15 mM, preferably about 5 to 10 mM, for LiCl, about 10 to 30 ng/ml, preferably about 15 to 25 ng/ml, for Shh, and about 1 to 40 ng/ml, preferably about 5 to 20 ng/ml, for IGF-1.

In this cultivation, PDGFRα-positive mesodermal cells are seeded to a culture vessel known per se (e.g., gelatin- or collagen-coated 10 cm cell culture dish and the like) to obtain a cell density of, for example, about 3 to 10×10⁴ cells/mL, preferably about 4 to 8×10⁴ cells/mL (about 3 to 10×10⁵ cells/10 cm dish, more preferably about 4 to 8×10⁵ cells/10 cm dish), and cultured in an incubator in an atmosphere of 5% CO₂/95% air at about 30 to 40° C., preferably about 37° C., for about 1 to 7 days, preferably about 2 to 4 days. Wnt signal inducer such as LiCl may also be added to a culture medium during induction of differentiation from pluripotent stem cells to PDGFRα-positive mesodermal cells. Preferably, Wnt signal inducer is added to a culture medium day 0 to day 3, more preferably day 1 or day 2 from the beginning of the induction of differentiation of pluripotent stem cells.

The fact of differentiation into skeletal muscle progenitor cells can be confirmed by, for example, analyzing the expression of the transcription factors Myf5 and MyoD by RT-PCR and the like. As required, furthermore, the expression of other transcription factors and cell surface antigens can also be examined. Examples of other transcription factors include Pax3 and Pax7. Examples of cell surface antigens include SM/C-2.6 and PDGFRα.

Because the PDGFRα-positive cell fraction obtained by this differentiation induction step expresses Myf5 and MyoD, whereas the PDGFRα-negative cell fraction does not express MyoD, it can be thought that most skeletal muscle progenitor cells are contained in the PDGFRα-positive fraction. Therefore, in a preferred embodiment of the present invention, a more highly purified skeletal muscle progenitor cell population can be obtained by selecting and separating a PDGFRα-positive cell fraction from a cell culture obtained by this differentiation induction step. Because the PDGFRα-negative cell fraction expresses Pax3 and Pax7, as well as Sox1, a marker of neurons in the developmental stage, it is thought to have differentiated into nervous cells; the non-fractionated cell population obtained by this induction step is potentially preferably useful as a source of graft cells, for example, when nerve regeneration is required in addition to skeletal muscle regeneration.

As stated above, in a second preferred aspect of the present invention, LiCl, Shh and IGF-1 are present in the Medium B. Accordingly, the present invention also provides a reagent kit for induction of differentiation from a PDGFRα-positive mesodermal cell to a skeletal muscle progenitor cell, comprising LiCl, Shh and IGF-1. These ingredients may be supplied in a state dissolved in water or an appropriate buffer solution, and may also be supplied as a dried (lyophilized) powder which may be used after being freshly dissolved in an appropriate solvent. These ingredients may be supplied as individual reagents in respective kits, and, as far as they do not adversely affect each other, they can be supplied as a single mixed reagent of 2 kinds or more.

(4) Cell Population Containing Skeletal Muscle Progenitor Cells Derived from Pluripotent Stem Cells

The present invention also provides a cell population containing skeletal muscle progenitor cells derived from pluripotent stem cells, produced by the foregoing step 2). The cell population may be a purified population of skeletal muscle progenitor cells, and 1 kind or more of cells other than skeletal muscle progenitor cells may be co-present. Here, “a skeletal muscle progenitor cell” is defined as a cell that is both Myf5-positive and MyoD-positive. As stated above, cells that are Myf5-positive and MyoD-positive are contained only in the PDGFRα-positive cell fraction, and the great majority of PDGFRα-positive cells are also positive for SM/C-2.6; therefore, purified skeletal muscle progenitor cells can be obtained by sorting out the cell culture obtained in the foregoing step 2), using an anti-PDGFRα antibody and/or anti-SM/C-2.6 antibody.

Preferably, the cell population containing skeletal muscle progenitor cells of the present invention is a cell population of iPS cell or ES cell derivation produced through the foregoing steps 1) and 2). When the iPS cell has been produced by, for example, transferring a reprogramming gene to a somatic cell by means of a retrovirus vector or lentivirus vector, the reprogramming gene is integrated in the genome of the cell; therefore, the skeletal muscle progenitor cells derived from the iPS cell also have the reprogramming gene integrated in the genome thereof. Because the skeletal muscle progenitor cells derived from iPS cells have been established for the first time by the present invention, the skeletal muscle progenitor cells having an extraneous reprogramming gene integrated in the genome thereof are of course novel cells. A reprogramming gene to be integrated in the genome of skeletal muscle progenitor cells is a nucleic acid that encodes one of the nuclear reprogramming substances described above with respect to preparing iPS cells, preferably 3 genes consisting of Oct3/4, Sox2, and Klf4, or 4 genes consisting of the foregoing three and c-Myc.

(5) Applications of Skeletal Muscle Progenitor Cells Derived from a Pluripotent Stem Cell

The skeletal muscle progenitor cell derived from pluripotent stem cells thus established can be used for varied purposes. For example, the cell enables a stem cell therapy by autologous or allogeneic transplantation in which skeletal muscle progenitor cells differentiated from an iPS cell induced using a somatic cell collected from a muscular disease patient or another person having the same or substantially the same type of HLA are transplanted to the patient to regenerate skeletal muscles. Furthermore, skeletal muscle progenitor cells differentiated from an iPS cell of a muscular disease patient are believed to reflect the status of muscle cells in the actual patient's body more than do the corresponding existing cell line, they can also be suitably used in in vitro, evaluation systems for the pharmacological efficacy and toxicity of therapeutic drugs for muscular diseases. They can further be preferably used as a tool for pathological research into muscular diseases of unknown causes.

Use of the skeletal muscle progenitor cell of pluripotent stem cell origin of the present invention in regenerative medicine is described in detail below.

As shown in an Example below, when the skeletal muscle progenitor cell of the present invention was transplanted to muscular dystrophy model mice, many muscle fibers were observed, and an inflammation suppressing effect and muscle tissue repair effect were observed. Also observed was induction of differentiation into satellite cells. Furthermore, because an inflammation-suppressing effect was observed not only by intramuscular injection, but also by intravenous injection, the skeletal muscle progenitor cell promotes skeletal muscle regeneration and satellite cell formation in muscular dystrophy and other various muscular diseases, and is useful in treating the diseases.

Examples of muscular diseases that can be treated with the skeletal muscle progenitor cell of the present invention include, but are not limited to, muscular dystrophy [e.g., Duchenne's muscular dystrophy (DMD), Becker type muscular dystrophy, congenital muscular dystrophy, limb-girdle muscular dystrophy, myotonic muscular dystrophy and the like], hereditary myopathies such as congenital myopathy, distal myopathy and mitochondrial diseases, non-hereditary myopathies such as multiple myositis, dermatomyositis and myasthenia gravis, neurogenic muscular diseases such as spinal amyotrophy, bulbar amyotrophy and amyotrophic lateral sclerosis, and the like. Preferably, the skeletal muscle progenitor cell of the present invention can be used to promote skeletal muscle regeneration and/or satellite cell formation in the treatment of myogenic diseases, particularly intractable hereditary and non-hereditary myogenic diseases, including progressive myodystrophies such as DMD.

As a skeletal muscle progenitor cell for treating muscular dystrophy and other hereditary muscular diseases, a skeletal muscle progenitor cell differentiated from a pluripotent stem cell induced from a person, other than the patient, having the same or substantially the same type of HLA as the patient's, is preferably used. In human regenerative medicine, it is difficult to obtain human ES cells having the same or substantially the same type of HLA; therefore, it is preferable to use a human iPS cell as a pluripotent stem cell for inducing a skeletal muscle progenitor cell.

In another embodiment, it is possible to use a skeletal muscle progenitor cell differentiated from an iPS cell derived from a somatic cell of the patient, as a skeletal muscle progenitor cell for the treatment of a hereditary muscular disease. For example, since an iPS cell induced from a somatic cell of a DMD patient lacks the dystrophin gene, the normal dystrophin gene is transferred to the iPS cell. The dystrophin cDNA is 14 kb in total length, and the adeno-associated virus (AAV) vector, which is best suited for transfection to muscle cells, can only accommodate a length of up to about 4.5 kb. For this reason, current strategic attempts of gene therapy include transfer of a shortened functional dystrophin gene [micro-dystrophin gene (3.7 kb)] using the AAV vector, transfer of a 6.4 kb mini-dystrophin gene using a retrovirus/lentivirus vector enabling insertion of a larger DNA, or transfer of the full-length dystrophin gene in a bare state or using a Gutted adenovirus vector. In case of an iPS cell, the highest transfer efficiency is achieved using retrovirus/lentivirus, but a full-length cDNA can be transferred using an artificial chromosome; this offers an advantage of a broader range of choices of gene therapy. In case of limb-girdle muscular dystrophy, which is caused by an abnormality of the sarcoglycan gene, the gene may be transferred to the iPS cell. Alternatively, the mutated site in the causal gene can be repaired on the basis of the endogenous DNA repair mechanism of the iPS cell or homologous recombination. Specifically, a chimeric RNA/DNA oligonucleotide (chimeraplast) having the normalized sequence at the mutated site is transferred and allowed to bind to the target sequence and form a mismatch, whereby the endogenous mechanism for DNA repair is activated to induce gene repair. Alternatively, gene repair can also be achieved by transferring a 400-800-base single-stranded DNA that is homologous to the mutated site to cause homologous recombination. The thus-obtained iPS cell with the repaired causal gene is induced to differentiate into a skeletal muscle progenitor cell via the foregoing steps 1) and 2), whereby a normal skeletal muscle progenitor cell derived from the patient can be produced.

Because any patient with a hereditary muscular disease essentially lacks normal gene products, however, an immune response to a normal gene product (e.g., dystrophin) can occur even when the patient's own skeletal muscle progenitor cell is used. In all cases, it seems necessary to use an immunosuppressant concurrently in transplanting skeletal muscle progenitor cells. Alternatively, to avoid this immune response, in case of DMD, the eutrophin gene, a dystrophin homologue also expressed in the patient's skeletal muscles, may be transferred as a substitute for the dystrophin function.

Meanwhile, in non-hereditary muscular diseases, the skeletal muscle progenitor cell differentiated from an iPS cell induced from a somatic cell of the patient is possibly a normal cell; therefore, the skeletal muscle progenitor cell can sometimes be used directly as a graft to the patient.

The cell population containing skeletal muscle progenitor cells obtained by the foregoing step 2) can be prepared as a preparation of purified cells obtained by sorting skeletal muscle progenitor cells, and can also be prepared as a preparation as it is without sorting. Although sorting enables a dose reduction, unsorted use is expected to offer advantages such as labor saving, cost reduction and the like. As stated above, of the cell population containing skeletal muscle progenitor cells obtained by the foregoing step 2), the PDGFRα-negative fraction is considered to be a cell population of the nervous system; therefore, this fraction can be effective in the treatment of diseases in which not only skeletal muscle regeneration, but also nerve regeneration is required, for example, neurogenic muscular diseases.

The skeletal muscle progenitor cells (including a cell population containing skeletal muscle progenitor cells; the same applies below) of the present invention are produced as a parenteral preparation, preferably as an injection, suspension, or drip infusion, in a mixture with a pharmaceutically acceptable carrier, by a conventional means. Examples of the pharmaceutically acceptable carrier that can be contained in the parenteral preparation include aqueous liquids for injection, such as physiological saline and isotonic solutions containing glucose and other auxiliary drugs (e.g., D-sorbitol, D-mannitol, sodium chloride and the like). The agent of the present invention may be formulated with, for example, a buffering agent (e.g., phosphate buffer solution, sodium acetate buffer solution), a soothing agent (e.g., benzalkonium chloride, procaine hydrochloride and the like), a stabilizer (e.g., human serum albumin, polyethylene glycol and the like), a preservative, an anti-oxidant and the like.

When the agent of the present invention is prepared as an aqueous suspension, skeletal muscle progenitor cells are suspended in one of the aforementioned aqueous liquids to obtain a cell density of about 1.0×10⁶ to about 1.0×10⁷ cells/mL.

Because the preparation thus obtained is stable and less toxic, it can be safely administered to mammals such as humans. Although the method of administration is not particularly limited, the preparation is preferably administered by injection or drip infusion. Useful routes of administration include intravenous administration, intra-arterial administration, intramuscular administration (topical administration to affected site) and the like. As shown in an Example below, the agent of the present invention is capable of selective engrafting at a site of muscle damage, and exhibiting inflammation suppressive action and skeletal muscle regeneration action equivalent to those by topical intramuscular administration, even when systemically administered by, for example, intravenous administration. Therefore, the agent of the present invention is preferably administered using a systemic route for administration such as intravenous administration or intra-arterial administration, particularly when symptoms are manifested in many sites. The dose of the agent of the present invention varies depending on the subject of administration, target site, symptoms, method of administration and the like. For a DMD patient (assuming a 60 kg body weight), in the case of intravenous administration, for example, it is usually convenient to administer the agent in an amount of about 1.0×10⁵ to about 1×10⁷ cells, based on the amount of skeletal muscle progenitor cells per dose, about 4 to about 8 times at about 1- to 2-week intervals.

The present invention is hereinafter described in further detail by means of the following examples, to which, however, the invention is never limited.

### EXAMPLES

### Reagents and Methods

### 1. Cultivation of Mouse iPS Cells and Induction of Differentiation

The three kinds of mouse iPS cells shown in (1)-(3) below were used.

(1) iPS-Nanog-20D-17, obtained by infecting mouse MEF with the 4 genes consisting of Oct3/4, Klf4, Sox2 and c-Myc by means of retrovirus [Okita, K. et al., Nature 448, 313-317 (2007)]
(2) iPS-DsRed, obtained by infecting mouse TTF with the 3 genes consisting of Oct3/4, Klf4 and Sox2 by means of retrovirus [Nakagawa, M. et al., Nat. Biotechnol., 26, 101-106 (2008)]
(3) Plasmid-iPS, obtained by transfecting mouse MEF with the 4 genes consisting of Oct3/4, Klf4, Sox2 and c-Myc by means of plasmid [Okita, K. et al., Science, 322, 949-953 (2008)]

The mouse iPS cells were cultured in the absence of feeder cells by the method of Takahashi et al. [Takahashi, K. and Yamanaka, S., Cell 126, 663-676 (2006)] with a minor modification. In summary, a basal medium was prepared by adding 2 mM L-glutamine (Nacalai Tesque), lxNon-essential amino acid (Invitrogen), 100 μM 2-mercaptoethanol (Invitrogen), 50 mU/L penicillin and 50 μg/L streptomycin to DMEM (Nacalai Tesque), and this was supplemented with fetal bovine serum (Invitrogen) at 15% to obtain an iPS cell maintenance medium. Subsequently, 10 mL of the maintenance medium under warming at 37° C. was added to a 10 cm cell culture dish, previously coated with 0.1% gelatin; 10 μL of LIF (ESGRO) was added, and 600,000 iPS cells were seeded and cultured in an incubator adjusted to 37° C., 5% CO₂, and 100% humidity. Two days later, the cells that had proliferated about 10 folds were subcultured. The following day, the medium was replaced with a fresh supply of the maintenance medium supplemented with LIF.

Differentiation of iPS cells was induced using a serum-free basal medium for differentiation induction prepared by adding 0.2% bovine serum albumin (Sigma), 100 μM 2-mercaptoethanol (Invitrogen), 50 mU/L penicillin and 50 μg/L streptomycin to S-Clone SF-03 (Sanko Junyaku Co., Ltd.). Various growth factors were added to the medium, and their effects were examined. Eventually, the following growth factors were used at the concentrations indicated.

| (For first 3 days) | | |
|---|---|---|
| BMP4 (Peprotech) | 10 | ng/ml |
| Activin A (Peprotech) | 5 | ng/ml |
| IGF-1 (Peprotech) | 10 | ng/ml |
| (For last 3 days) | | |
| LiCl (Wako) | 5 | mM |
| Sonic Hedgehog (R&D) | 20 | ng/ml |
| IGF-1 (Peprotech) | 5 | ng/ml |

Undifferentiated cells were detached from the dish as in the passage subculture, and twice washed with the differentiation induction basal medium. 10 ml of the culture medium for the first 3 days was added to a 10 cm cell culture dish coated with collagen type IV (Nitta Gelatin), and 500,000 undifferentiated iPS cells were seeded thereinto. The cells were cultured in an incubator adjusted to 37° C., 5% CO₂, and 100% humidity for 3 days, after which the medium was replaced with the culture medium for the last 3 days. The cells were thus differentiation-induced for a total of 6 days, from which skeletal muscle progenitor cells were separated by the method of cell separation described below, and these were used in the subsequent experiments.

### 2. Cell Separation

The mouse iPS cells induced by the above-described differentiation induction method were divided into a population of skeletal muscle progenitor cells and a population of other cells using FACS Aria (Becton Dickinson). The cells were stained using antibodies as described previously [Sakurai, H. et al., Stem Cells 24, 575-586 (2006)]. Three different rat monoclonal antibodies were used: APA5 (anti-PDGFRα), ECCD2 (anti-ECD) and SM/C-2.6. The former two were supplied by Dr. Nishikawa [Sakurai, H. et al., Stem Cells 24, 575-586 (2006)], and the remaining one by Dr. Yamamoto [Fukada, S. et al., Exp Cell Res., 296, 245-255 (2004)]. The APA5 and SM/C-2.6 were conjugated with biotin (PIERCE) and fluorescently labeled using streptavidin-APC as a secondary antibody. The ECCD2 was directly conjugating with Alexa488 (Molecular Probes) by a conventional method and fluorescently labeled. The fluorescently labeled cells were suspended in Hanks' balanced salt solution (Invitrogen) supplemented with 1% bovine serum albumin to obtain a cell density of 5,000,000 cells per mL, fluorescence was examined and analyzed using FACS Aria, and the PDGFRα-positive fraction was separated and recovered.

### 3. Gene Expression Analysis

RNA was extracted from the recovered cells using Sepasol Reagent (Nacalai Tesque). cDNA was synthesized from the RNA using the SuperScriptll reverse transcription kit (Invitrogen). DNA was amplified by 25-35 cycles of PCR reaction using the ExTaq PCR reagent kit (Takara Bio) and the PTC200 thermal cycler (DNA engine), and analyzed by electrophoresis. The PCR primers shown below were used.

β-Actin:
(SEQ ID NO: 1)
Fw: 5′-AGTGTGACGTTGACATCCGT-3′
(SEQ ID NO: 2)
Rv: 5′-GCAGCTCAGTAACAGTCCGC-3′
PDGFRα:
(SEQ ID NO: 3)
Fw: 5′-CTTTGTGCCTCTCGGGATGA-3′
(SEQ ID NO: 4)
Rv: 5′-AGGTTACTTGAGTCTCCGGATCT G-3′
Pax3:
(SEQ ID NO: 5)
Fw: 5′-CTGCACTCAAGGGACTCCTC-3′
(SEQ ID NO: 6)
Rv: 5′-GTTGTCACCTGCTTGGGTTT-3′
Pax7:
(SEQ ID NO: 7)
Fw: 5′-CCGTGTTTCTCATGGTTGTG-3′
(SEQ ID NO: 8)
Rv: 5′-ACCAGAAGGAGCAGCACTGT-3′
Myf5:
(SEQ ID NO: 9)
Fw: 5′-GAGTTTGGGGACCAGTTTGA-3′
(SEQ ID NO: 10)
Rv: 5′-GCTTCAGGGCTTCTTTTCCT-3′
MyoD:
(SEQ ID NO: 11)
Fw: 5′-TACCCAAGGTGGAGATCCTG-3′
(SEQ ID NO: 12)
Rv: 5′-CTGGGTTCCCTGTTCTGTGT-3′
Sox1:
(SEQ ID NO: 13)
Fw: 5′-GCCCAGGAAAACCCCAAGATG-3′
(SEQ ID NO: 14)
Rv: 5′-CCGTTAGCCCAGCCGTTGAC-3′

### 4. Transplantation to Mice

The mice used in the experiment of induction of muscle regeneration with cardiotoxin were of the C57BL/6 line (Japan SLC). In the experiment with a mouse model of muscular dystrophy, DMD-null mice [kindly supplied by Dr. Hanaoka at the Faculty of Sciences in Kitasato University; Kudoh, H. et al., Biochem Biophys Res Commun., 328, 507-516 (2005)] were used. The animal transplantation experiments were performed in compliance with the “Regulation on Animal Experimentation at Kyoto University”.

Cardiotoxin was administered 3 days before transplantation. Under diethyl ether anesthesia, 50 μL of 10 μM cardiotoxin (Wako) was administered to the left tibialis anterior muscle by intramuscular injection. Three days later, 500,000 to 1,000,000 skeletal muscle progenitor cells, separated using FACS Aria, were suspended in 50 μl of PBS and maintain-cultured at 37° C. Subsequently, again under diethyl ether anesthesia, the cells were transplanted to the left tibialis anterior muscle by intramuscular injection, or transplanted from the orbital venous plexus by venous injection.

In the transplantation experiment using the DMD-null mice, 2,000,000 skeletal muscle progenitor cells, separated using FACS Aria, were suspended in 100 μl of PBS, maintain-cultured at 37° C., and transplanted to both tibialis anterior muscles at 50 μl for each side (1,000,000 cells for each side) by intramuscular injection under diethyl ether anesthesia.

In both the experiments, 28 days later, mice were euthanized with carbon dioxide, the tibialis anterior muscle, as well as the quadriceps femoris muscle, forefoot extensors, and diaphragm were cut off, and rapidly frozen by immersion in isopentane (Nacalai Tesque) under cooling with liquid nitrogen. Each tissue section sample obtained was sliced into sections 10 μm thick using a cryostat (Leica), and allowed to adhere to APS-coated glass slides (Matsunami Glass) and dried.

### 5. Fluorescent Immunostaining

Each tissue section prepared was fixed in 4% para-formaldehyde (Nacalai Tesque)/PBS at room temperature for 20 minutes, and washed with PBS for 5 minutes 3 times, after which blocking was performed using a PBS supplemented with 1% goat serum (Sigma), 0.1% bovine serum albumin, and 0.2% Triton X-100 (Nacalai Tesque) at room temperature for 1 hour. Primary antibodies were used after being diluted to concentrations of 1:500 for anti-DsRed (Rabbit Polyclonal: CLONTECH Laboratories), 1:150 for anti-Laminin-α2 (Rat Monoclonal: ALEXIS), 1:200 for anti-Dystrophin (Mouse Monoclonal: Sigma), and 1:200 for anti-SM/C-2.6 (Rat Monoclonal: provided by Dr. Yamamoto), in the aforementioned blocking liquid. The reaction was carried out at 4° C. for 16-18 hours. After the glass slide was washed with a PBS supplemented with 0.2% Triton X-100 (PBST) 3 times, the sample was reacted with the secondary antibodies: anti-Rabbit IgG-PE conjugated, anti-Rat IgG-Alexa647 conjugated, anti-Mouse IgG-Alexa647 conjugated, and Streptavidin-Alexa488 conjugated (all are products of Molecular Probes), all diluted at 1:500 in PBST, at room temperature for 2 hours. Anti-Pax7 (mouse monoclonal: R&D) was conjugated directly with each secondary antibody using the Zenon-Alexa488 IgG1 labeling kit (Molecular Probes), and the conjugate was diluted to a concentration of 1:100 in a PBS supplemented with 0.2% Triton X-100, and reacted at room temperature for 1 hour. Subsequently, to stain the cell nuclei, 5 μg/ml DAPI (Sigma) was diluted in PBST 5000 folds, and the dilution was reacted at room temperature for 5 minutes and washed with PBS 3 times, after which cover glass was placed on the glass slide, and the sample was sealed. The stained tissue section was examined for data acquisition using the SP5 confocal microscopic system (Leica).

### 6. Mature Skeletal Muscle Differentiation Induction

The PDGFRα-positive and -negative fractions of iPS cells on day 6 of differentiation, which were separated by FACS Aria, were seeded again into collagen type I coat 24 well dish (IWAKI) and cultured. The number of cells were 200,000 per well. As the medium, S-clone SF-03 (Sanko Junyaku) supplemented with 0.2% bovine serum albumin (Sigma Ltd.), 100 μM 2-mercaptoethanol (Invitrogen), 50 mU/L Penicillin/50 μg/L Streptomycin was used as a differentiation induction basic medium, which was further added with the following growth factors. To remove dead cells, the medium was exchanged with a medium having the same composition 24 hr from the start of the differentiation induction.

| (for first 4 days) | | |
|---|---|---|
| HGF (R&D) | 10 | ng/ml |
| bFGF (Peprotech) | 2 | ng/ml |
| IGF-1 (Peprotech) | 2 | ng/ml |
| (for last 3 days) | | |
| IGF-1(Peprotech) | 2 | ng/ml |

### 7. Immunostaining of Cultured Cells

The cells differentiated into mature skeletal muscle by the above-mentioned differentiation induction were evaluated by immunostaining. The medium was discarded leaving the cells attached to the dish, and the cells were fixed in 4% para-formaldehyde (Nacalai Tesque)/PBS at 4° C. for 10 minutes, and washed with PBS for 5 minutes 3 times, after which blocking was performed using a PBS supplemented with 1% goat serum (Sigma), 0.1% bovine serum albumin, and 0.2% Triton X-100 (Nacalai Tesque) at room temperature for 1 hour. Primary antibodies were used after being diluted to concentrations of 1:200 for anti-Myogenin (Rabbit Polyclonal: Santa Cruz Biotechnology) in the aforementioned blocking liquid. The reaction was carried out at 4° C. for 16-18 hours, and washed with a PBS supplemented with 0.2% Triton X-100 (PBST) 3 times. The sample was reacted with the secondary antibodies anti-Rabbit IgG-HRP conjugated (Vector), diluted at 1:200 in PBST, at room temperature for 2 hr. After washing 3 times with PBS, color development was performed 3 min using HRP color development kit (Dako). After washing with PBST, the cells were observed with All-in-One microscope BioZero (KEYENCE) and photographed. After photographing, the nucleus was stained with a Giemsa staining solution (Merck) at room temperature for 10 min, washed 3 times with PBS, observed with All-in-One microscope BioZero (KEYENCE) and photographed. The positive cells were measured by analysis based on visual observation of the whole well.

### Example 1

### Induction of Differentiation from iPS Cells to Primitive Streak Mesodermal Cells

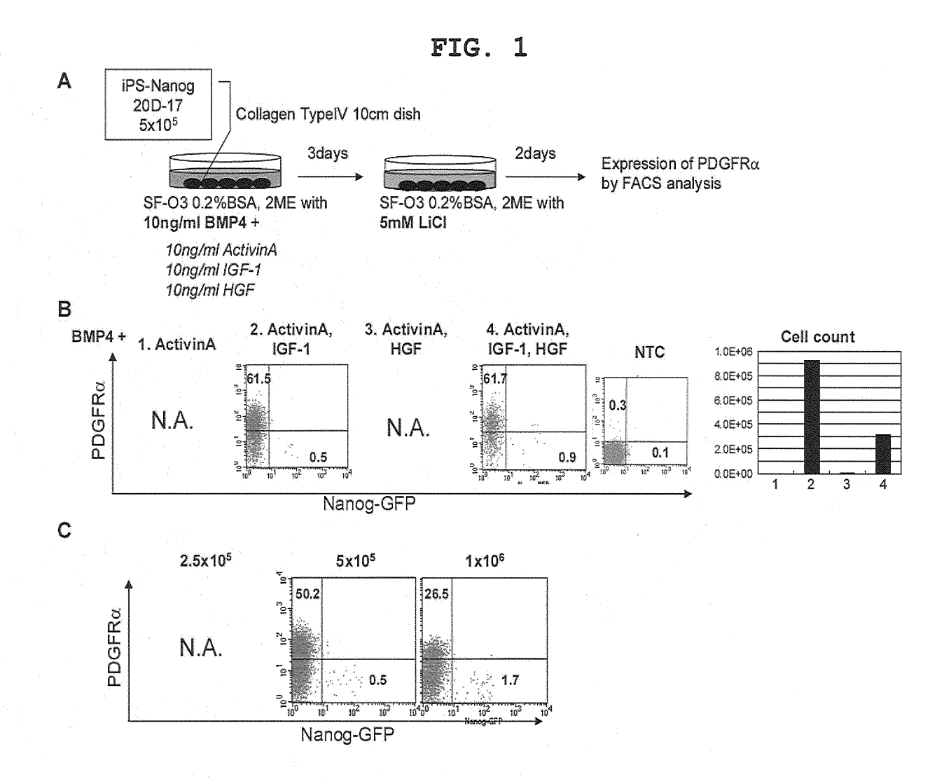

An attempt was made to induce the differentiation of iPS-Nanog-20D-17, iPS-DsRed and Plasmid-iPS by the method of serum-free differentiation induction using BMP4 and LiCl established in a study using mouse ES cells (Japanese Patent Application No. 2008-186348; the method indicated by bald letters in FIG. 1A), but no cells survived. With this in mind, a first experiment was performed to determine whether the addition of growth factors other than BMP4 supports the survival of iPS cells and promotes their differentiation into primitive streak mesodermal cells (hereinafter sometimes simply referred to as “mesoderm”). Activin A, IGF-1, and HGF were examined as growth factor candidates (FIG. 1A, italicized). With Activin A added to all culture conditions examined, 4 conditions were analyzed: “neither IGF-1 nor HGF was added”, “IGF-1 was added alone”, “HGF was added alone”, and “both were added”. The iPS cell used was iPS-Nanog-20D-17. The results are shown in FIG. 1B. Cell proliferation was observed in the presence of IGF-1 (FIG. 1B, 2 and 4), with little cells surviving under other conditions (some cells survived in the presence of Activin A and absence of IGF-1 and HGF, although their count was smaller than that with the addition of IGF-1; data not shown). An evaluation of the degree of mesoderm differentiation with the expression of PDGFRα as an index revealed that the differentiation rate was 61.5% for the IGF-1-alone addition group, and 61.7% for the IGF-1-HGF-concurrent addition group; a similar degree of differentiation into mesoderm was observed (FIG. 1B). However, the viable cell count was about 3 times higher in the IGF-1-alone addition group than in the IGF-1-HGF-concurrent addition group (FIG. 1B, right). These results showed that in the induction during the starting 3 days, the highest induction efficiency was obtained when three kinds of growth factors consisting of BMP4, Activin A, and IGF-1 were added simultaneously.

Also examined was the influence of seeded cell density on mesoderm differentiation as of the time of differentiation induction (FIG. 1C). The results showed that the highest induction efficiency was obtained, with the percentage of PDGFRα-positive cells being 50.2%, when 500,000 cells were seeded to a 10 cm dish. At lower numbers of cells seeded, few cells survived; at a higher number of cells (1,000,000 cells), the percentage of PDGFRα-positive cells was 26.5%, representing an about half induction efficiency (FIG. 1C).

### Example 2

### Influences of BMP4 and Activin A Concentrations in Induction of Differentiation from iPS Cells to Primitive Streak mesodermal cells

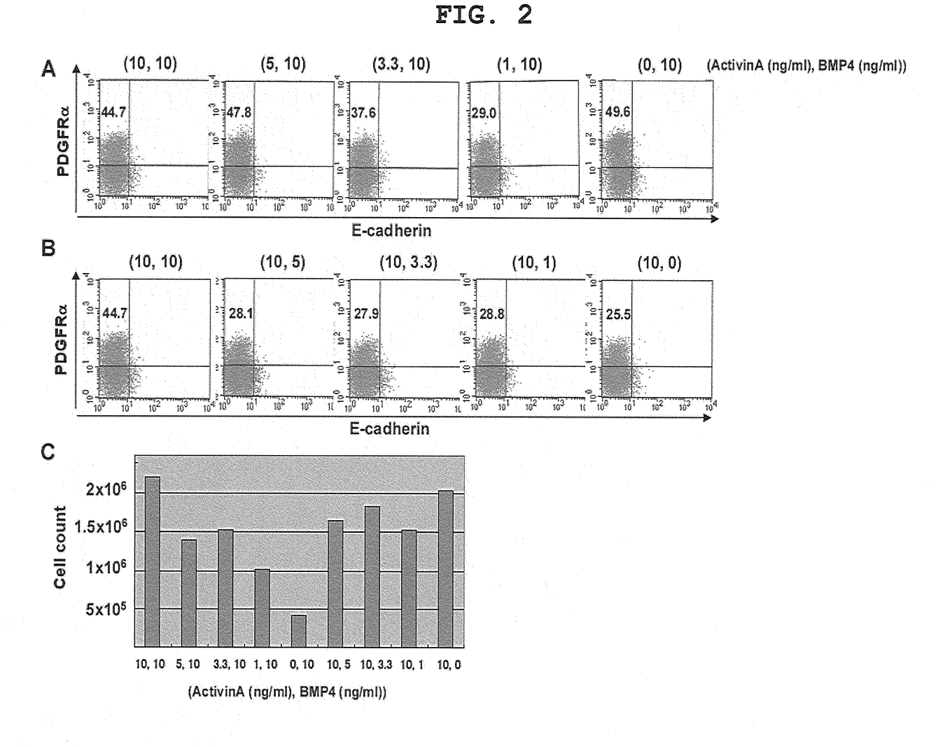

The influences of the concentrations of BMP4 and Activin A in the induction of differentiation from iPS cells to primitive streak mesodermal cells were examined. Specifically, of the addition conditions of Activin A 10 ng/ml, BMP4 10 ng/ml, and IGF-1 10 ng/ml determined in the previous section, the concentration of Activin A alone (FIG. 2A) or BMP4 alone (FIG. 2B) was changed, and the expression of PDGFRα was evaluated by FACS.

Analysis of the influence of Activin A concentration showed that the percentage of PDGFRα-positive cells induced exceeded 40% at concentrations of 5 ng/ml or more, but the percentage decreased dose-dependently at lower concentrations. In the absence of Activin A, the percentage was 49.6%, demonstrating high induction efficiency. However, comparing the viable cell counts obtained, the highest viable cell count was obtained under the conditions involving the addition of Activin A at 10 ng/ml, and the viable cell count tended to decrease with the reduction of Activin A concentration; the cell count decreased to an about one-sixth level for Activin A 0 ng/ml compared with 10 ng/ml (FIG. 2C). Hence, it was found that in the absence of Activin A, a fewer PDGFRα-positive cells were obtained because the overall cell count decreased, although the percentage of cells induced was high. Based on these findings, Activin A was thought to be more effective when added at concentrations of about 5-10 ng/ml.

Regarding the influence of BMP4 concentration, the highest percentage of PDGFRα-positive cells of 44.7% was obtained when BMP4 was added at 10 ng/ml; at all concentrations of 5 ng/ml or less, the ratio decreased to about 25-28% (FIG. 2B). This result showed that a threshold exists for the influence of BMP4, with the efficiency of induction of differentiation into mesoderm decreasing at BMP4 concentrations of 5 ng/ml or less. It was also found that even when the concentration of BMP4 decreased, the viable cell count obtained remained nearly constant (FIG. 2C).

These results showed that Activin A had a concentration-dependent effect on both differentiation efficiency and cell proliferation, and that BMP4 promotes mesoderm differentiation and does not have a contributory effect on cell proliferation at concentrations exceeding a certain threshold.

### Example 3

### Induction of Differentiation from Primitive Streak Mesodermal Cells Derived from iPS Cells to Skeletal Muscle Progenitor Cells

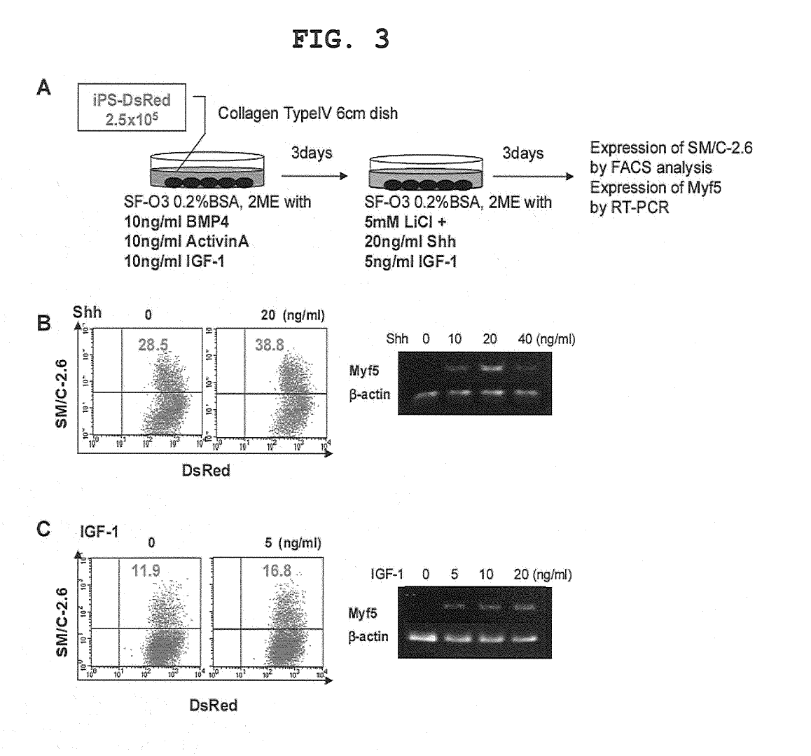

Although the results described in the previous section demonstrated that three factors consisting of Activin A, BMP4, and IGF-1 were essential in the differentiation from iPS cells to primitive streak mesodermal cells (during the first 3 days), the factor that promotes the differentiation from primitive streak mesodermal cells to skeletal muscle progenitor cells (during the last 3 days) remained unidentified. To find a method for effectively inducing the differentiation, the present inventors investigated the potential of the Wnt signal inducer LiCl, used alone or in combination with other growth factors, for inducing the differentiation of iPS cells into skeletal muscle progenitor cells. Selected growth factor candidates, i.e., Sonic Hedgehog (Shh) and IGF-1, were added to the differentiation medium for the last 3 days, and their influences on differentiation into skeletal muscle progenitor cells were examined (FIG. 3A). The iPS cell used was iPS-DsRed. The results are shown in FIG. 3B. The degree of differentiation into skeletal muscle progenitor cells was analyzed with the expression of SM/C-2.6 as an index. Also analyzed was the expression of Myf5, which is a skeletal muscle-specific transcription factor in the RNA as a whole. SM/C-2.6 is a marker capable of specifically staining satellite cells, which are skeletal muscle stem cells [Fukada, S. et al., Exp Cell Res., 296, 245-255 (2004)], and has been reported to enable the separation of skeletal muscle progenitor cells from a mouse ES cell induction system [Chang, H. et al., Generation of transplantable, functional satellite-like cells from mouse embryonic stem cells. Faseb J., 23: 1907-1919 (2009)].

First, the influence of the addition of Shh was determined. iPS-DsRed was induced to differentiate as illustrated in FIG. 3A. During the last 3 days, the cells were cultured in the presence of LiCl, alone or with the addition of Shh 20 ng/ml; the cells were induced to differentiate for a total of 6 days, stained with SM/C-2.6, and evaluated. As a result, the percentage of SM/C-2.6-positive cells was 28.5% in the Shh-free group, and 38.8% in the Shh 20 ng/ml addition group, showing an increase of about 10% (FIG. 3B). RT-PCR analysis of the expression of Myf5 revealed that the expression of Myf5 was not observed at all in the Shh-free group, whereas Myf5 was expressed in the Shh addition groups, with the highest expression observed at a concentration of 20 ng/ml (FIG. 3B).

As for the effect of IGF-1, an analysis of the expression of SM/C-2.6 in the same manner as with Shh revealed that the percentage increased from 11.9% in the IGF-1-free group to 16.8% in the IGF-1 5 ng/ml addition group (FIG. 3C). Regarding the expression of Myf5, no expression of Myf5 was observed in the IGF-1-free group. When IGF-1 was added, however, the expression of Myf5 was evident irrespective of the concentration of IGF-1 (FIG. 3C). These results demonstrate that LiCl alone is insufficient to induce the differentiation of iPS cells into skeletal muscle progenitor cells because of the lack of the expression of Myf5, and that addition of Shh or IGF-1 increases the number of SM/C-2.6-positive cells and promotes the expression of Myf5. The optimal concentration of Shh was determined at about 20 ng/ml; IGF-1 was shown to be effective when present at 5 ng/ml or more.

It was also confirmed that these methods of differentiation induction enable similar induction of skeletal muscle progenitor cells even in clones of Plasmid-iPS cells.

### Example 4

### Effect of Skeletal Muscle Progenitor Cells Derived from iPS Cells on Damaged Skeletal Muscles

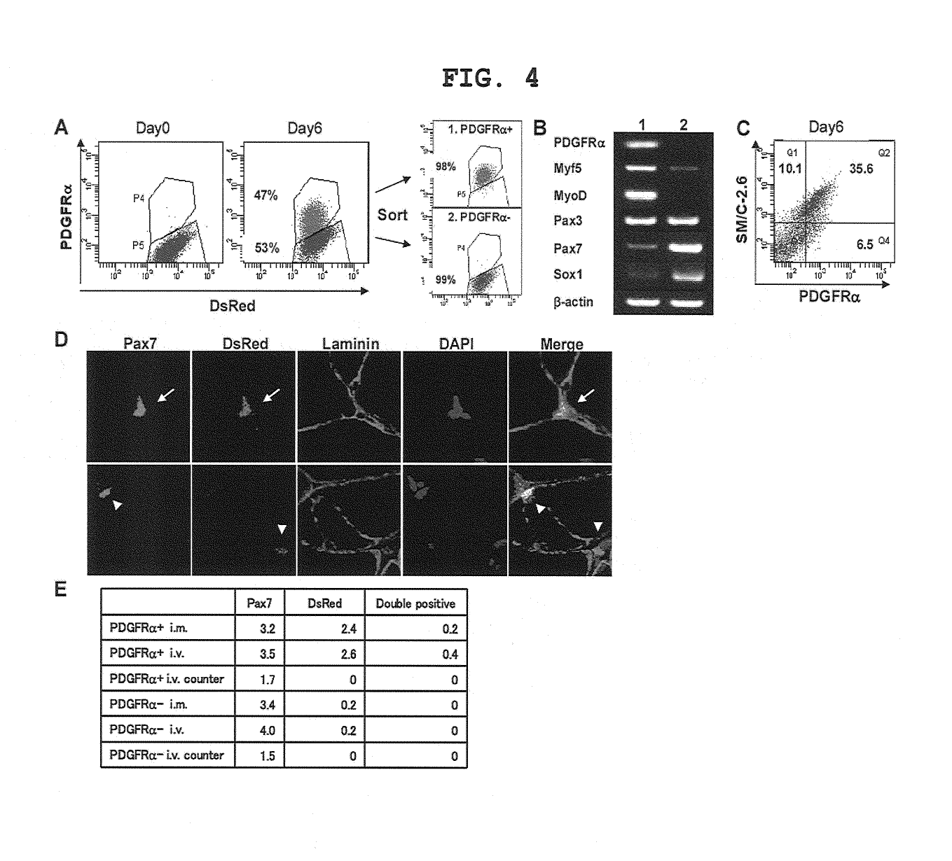

Experiments were performed to determine whether the mesodermal cell population obtained by differentiation induction by the above-described serum-free induction method actually contained skeletal muscle progenitor cells. Differentiation was induced by the method of differentiation induction established in Examples 1-3 (the method described in Section 1 under “Reagents and Methods”). On day 6 of differentiation, the cells were analyzed with PDGFRα as a marker; PDGFRα was expressed in about 47% of the cells (FIG. 4A). This cell population was divided by FACS Aria into a PDGFRα-positive fraction and a negative fraction. The separation efficiency for each fraction was as high as not lower than 98% (FIG. 4A, 1. for the PDGFRα⁺ fraction, 2. for the PDGFRα⁻ fraction). RNA was extracted from these two fractions, and the gene expression was analyzed by RT-PCR (FIG. 4B, 1 for PDGFRα⁺ fraction, 2 for PDGFRα⁻ fraction). Examination of the expression of PDGFRα as an index of separation efficiency revealed that PDGFRα was expressed only in the PDGFRα-positive fraction, demonstrating the appropriateness of the separation by FACS. Regarding the expression of Myf5 and MyoD, which are skeletal muscle-specific markers, Myf5 was considerably highly expressed in the PDGFRα-positive fraction, whereas MyoD was expressed only in the PDGFRα-positive fraction. This demonstrated that the cell population having characters of skeletal muscle progenitor cells are for the most part contained in the PDGFRα-positive fraction. Additionally, Pax3 and Pax7, which are markers of the dermomyotome, the developmental origin of skeletal muscle progenitor cells, were expressed in both the PDGFRα-positive and -negative fractions; rather, Pax7 was strongly expressed in the PDGFRα-negative fraction. However, since Pax3 and Pax7 are known to be expressed also in the early development of the nervous system, and also since the expression of Sox1, a marker of neurons in the developmental stage, was strongly expressed in the PDGFRα-negative fraction, it was thought to be likely that the Pax3 and Pax7 expressed in the PDGFRα-negative fraction represent a cell population of the nervous system. The expression of β-actin served for control to indicate a constant amount of RNA.

Comparing the expression of SM/C-2.6, a marker of skeletal muscle progenitor cells described in the previous section, and PDGFRα, all SM/C-2.6-positive cells were found to be present in the PDGFRα-positive to -weakly-positive fractions; it was thought that skeletal muscle progenitor cells are contained in the PDGFRα-positive fraction (FIG. 4C). These results demonstrated that the PDGFRα-positive fraction as of day 6 of differentiation induction was a cell population containing skeletal muscle progenitor cells, whereas the PDGFRα-negative fraction was a cell population free from skeletal muscle progenitor cells.

Subsequently, experiments were performed to determine whether this population of PDGFRα-positive cells have characters as skeletal muscle progenitor cells in vivo as well. The iPS cells used were iPS-DsRed cells. Because iPS-DsRed cells are derived from the TTF of a mouse that is constantly expressing DsRed, their presence serves as an index of the fact of iPS derivation.

PDGFRα-positive cells induced from an iPS-DsRed cell were transplanted by intramuscular injection to skeletal muscles of mice with muscle regeneration caused by cardiotoxin treatment. 4 weeks after the transplantation, the expression of DsRed was analyzed to evaluate the behavior of the cells in vivo (FIG. 4D). Studies have shown that PDGFRα-positive cells derived from mouse ES cells differentiate into satellite cells, which are stem cells of skeletal muscles, in damaged skeletal muscles [Sakurai, H. et al., Stem Cells 26, 1865-1873 (2008)]. With this in mind, whether the induced PDGFRα-positive cells have differentiated into satellite cells as reported was determined by evaluating the expression of Pax7, a satellite cell marker (FIG. 4D). The results showed that DsRed-positive cells derived from iPS cells differentiated into Pax7-positive cells deemed satellite cells inside the laminin and engrafted (FIG. 4D, upper panel, arrow). However, not all transplanted cells were Pax7-positive, but Pax7-negative DsRed-positive cells outside the laminin (FIG. 4D, lower panel, arrowhead in the 2nd leftmost panel) were also observed. For control, results for satellite cells from the graft recipient mice are also shown (FIG. 4D, lower row, arrowhead in the leftmost panel). These results demonstrated that PDGFRα-positive cells derived from iPS cells are capable of differentiating into satellite cells in damaged skeletal muscles.

Subsequently, methods of administration were examined. PDGFRα-positive cells derived from mouse iPS cells were transplanted to mice with cardiotoxin-treated damaged muscles not only by intramuscular injection, but also by intravenous injection; 4 weeks later, the tissue was analyzed (FIG. 4E). FIG. 4E shows mean numbers of positive cells visible at five sites counted at the time of tissue examination at a magnifying rate of x400. Whether the cells were transplanted by intramuscular injection (i.m.) or intravenous injection (i.v.), the number of DsRed-positive cells apparent per visual field remained almost constant (2.4 versus 2.6, FIG. 4E, 2 upper rows). Of the DsRed-positive cells derived from iPS cells, some were also Pax7-positive (double positive), with no major difference between intramuscular injection and intravenous injection (0.2 versus 0.4, FIG. 4E, 2 upper rows). This result led to the thought that the transplantation efficiency for PDGFRα-positive cells derived from iPS cells is constant between intramuscular injection and intravenous injection. Interestingly, in the tibialis anterior muscle of the hind leg not treated with cardiotoxin (counter), absolutely no DsRed-positive cells were observed (FIG. 4E, 3rd uppermost row). This was attributed to the fact that the skeletal muscle progenitor cells derived from iPS cells, even when spread to the whole body by intravenous injection, were engrafted selectively in skeletal muscles with a regeneration signal due to inflammation (FIG. 4E, 3rd uppermost row).

Meanwhile, little PDGFRα-negative cells were engrafted in the cardiotoxin-treated skeletal muscle, the number of the engrafted cells was about 0.2 cells/visual field (1 cell per 5 visual fields) for both intravenous injection and intramuscular injection. Engrafted cells were observed only in inflamed interstitium, and there were absolutely no Pax7-positive cells; it was thought that the PDGFRα-negative cells had not differentiated into a skeletal muscle pedigree, including satellite cells (FIG. 4E, 4th and 5th uppermost rows).

### Example 5

### Effect of Skeletal Muscle Progenitor Cells Derived from iPS Cells on Mouse Model of Muscular Dystrophy

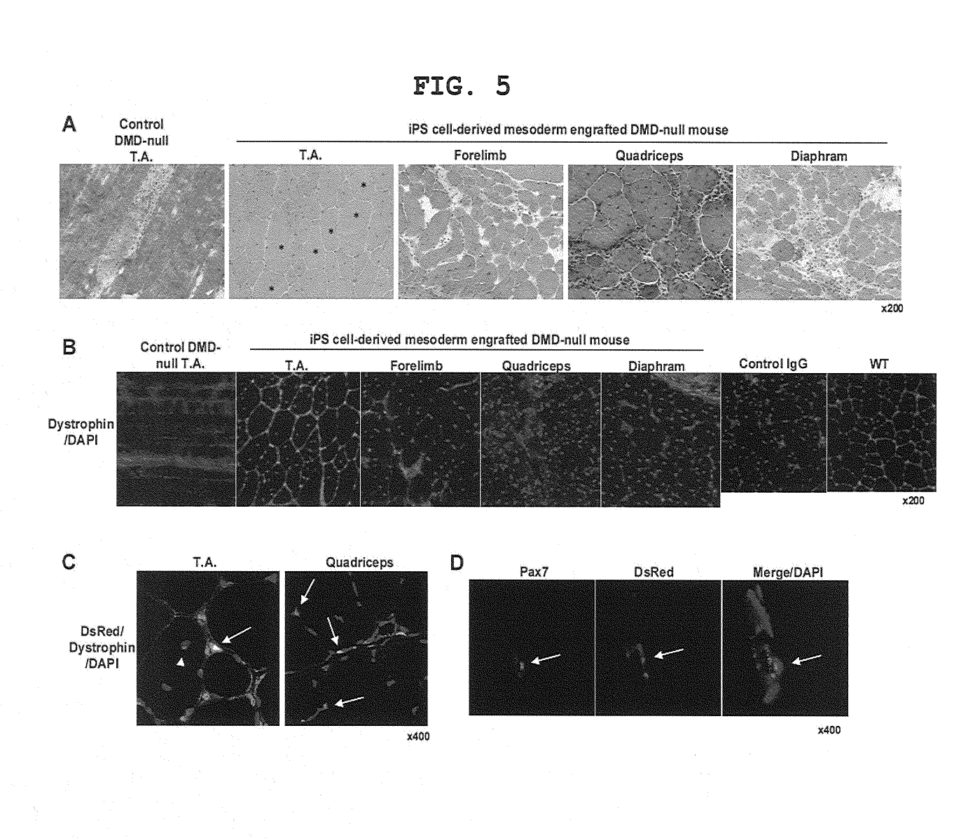

As described above, it was confirmed that mesodermal cells derived from iPS cells (skeletal muscle progenitor cells) differentiate into satellite cells when transplanted to cardiotoxin-treated mice. Subsequently, an analysis was performed to determine whether this is contributory to skeletal muscle regeneration in the bodies of muscular dystrophy model mice (DMD-null mice). On day 6 of differentiation, PDGFRα-positive cells derived from iPS cells were separated by FACS, and transplanted to the tibialis anterior muscle (T.A.) of 8-week old DMD-null mice by intramuscular injection. Four weeks later, the tissue was analyzed. In control DMD-null mice receiving no cells transplanted, the skeletal muscle interstitium was infiltrated by a very large number of inflammatory cells, with nuclei localized centrally, confirming that the skeletal muscles were amid regeneration (FIG. 5A, leftmost). Meanwhile, in the T.A. receiving skeletal muscle progenitor cells derived from iPS cells transplanted by intramuscular injection, almost no sign of interstitial inflammation was observed. Although some skeletal muscle nuclei were localized centrally, many muscle fibers were found to have their nuclei shifted to the peripheries, suggesting completion of regeneration (FIG. 5A, 2nd leftmost, marked with *). However, when other skeletal muscles of the mice receiving skeletal muscle progenitor cells derived from iPS cells transplanted by intramuscular injection were evaluated, severe interstitial infiltration of inflamed cells was evident, with nuclei localized centrally in the muscle fibers in almost all cases, in all the forelimbs, quadriceps femoris muscles, and diaphragm (FIG. 5A, three panels in the right from center). These results confirmed that in the muscular dystrophy model mice, transplantation of skeletal muscle progenitor cells derived from iPS cells to skeletal muscles suppresses inflammation at least in the intramuscular injection site, which in turn leads to completion of muscle regeneration.

Subsequently, to determine whether the above-described histological changes are due to the expression of dystrophin, fluorescent immunostaining of dystrophin was performed (FIG. 5B). As expected in control DMD-null mice, no expression of dystrophin (visualized in red-purple) was observed (FIG. 5B, leftmost). However, because Anti-Mouse IgG-Alexa647, used as a secondary antibody, recognized IgG in tissues rich in inflamed cells, an obscure red-purple signal was observed correspondingly in inflamed portions. Judging from the finding of a signal observed at the same site even in negative control samples with control IgG, it is evident that this result is attributed to the background of the secondary antibody (FIG. 5B, 2nd rightmost). In wild-type mouse positive controls, dystrophin was expressed in a mesh-like pattern around the peripheries of muscle fibers (FIG. 5B, rightmost). In the T.A. receiving skeletal muscle progenitor cells derived from iPS cells transplanted thereto, dystrophin was expressed in a mesh-like pattern around the peripheries of muscle fibers, as seen in the wild type (FIG. 5B, 2nd leftmost). When other skeletal muscles of the mice receiving skeletal muscle progenitor cells derived from iPS cells transplanted thereto were evaluated, a little expression of dystrophin in a mesh-like state was observed in all the forelimbs, quadriceps femoris muscles, and diaphragm, although a background corresponding to the inflamed tissue in the interstitium was observed. These results showed that skeletal muscle progenitor cells derived from iPS cells were engrafted as cells that produce muscle fiber dystrophin when transplanted to dystrophin-deficient muscular dystrophy model mice, resulting in the expression of dystrophin in the muscle fibers, whereby muscle fiber collapse is suppressed, the inflammation in the skeletal muscles is cured, and completion of the tissue repair and over-regeneration state is promoted.

To determine whether this tissue repair in the muscular dystrophy model mice is really by the action of the transplanted skeletal muscle progenitor cells derived from iPS cells, fluorescent immunostaining, including DsRed, was performed. As a result, in the T.A. receiving skeletal muscle progenitor cells derived from iPS cells transplanted thereto, DsRed was also expressed in the cytoplasm in some muscle fibers (FIG. 5C, left, arrowhead), although DsRed-positive cells were observed in the interstitium (FIG. 5C, left, arrow), and dystrophin was also expressed in the same site. Therefore, it was thought that the skeletal muscle progenitor cells derived from iPS cells contributed to the expression of dystrophin. Meanwhile, even in the quadriceps femoris muscles not receiving direct transplantation, DsRed-positive cells were observed in the interstitium (FIG. 5C, right, arrow). This result suggests that graft cells may migrate into blood vessels and spread to the whole body also when transplanted by intramuscular injection.

Furthermore, to determine whether the skeletal muscle progenitor cells derived from iPS cells that were transplanted to the muscular dystrophy model mice and engrafted were differentiated into satellite cells in vivo, fluorescent immunostaining, including Pax7 and DsRed, was performed (FIG. 5D). Some DsRed-positive cells were found to express Pax7 (FIG. 5D, arrow); it was demonstrated that the skeletal muscle progenitor cells derived from iPS cells differentiated into satellite cells even in transplantation to the muscular dystrophy model mice.

The motor function of DMD-null mice transplanted with skeletal muscle progenitor cells was also evaluated. A hanging test was performed to determine how long each mouse can endure grasping a cage mesh by its limbs upside down. The test was repeated 3 times at 5-minute resting intervals, and mean hanging time was calculated for a total of 3 repeats. The DMD-null mice not receiving the skeletal muscle progenitor cells at all exhibited a hanging time of about 2.3 seconds, whereas the mice receiving the skeletal muscle progenitor cells transplanted to the tibialis anterior muscle by intramuscular injection exhibited a hanging time of about 6.7 seconds; the hanging time extended about 3 folds. This result demonstrated that suppression of muscle tissue inflammation by transplantation led to an improvement of the motor function.

### Example 6

### Effect of Skeletal Muscle Progenitor Cells Derived from iPS Cells on Mouse Model of Muscular Dystrophy (2)

In Example 5, PDGFRα positive cells derived from iPS cells were transplanted to both T.A. of DMD-null mouse. In this Example, the same experiment as in Example 5 was performed except that the cells were transplanted to only one T.A.

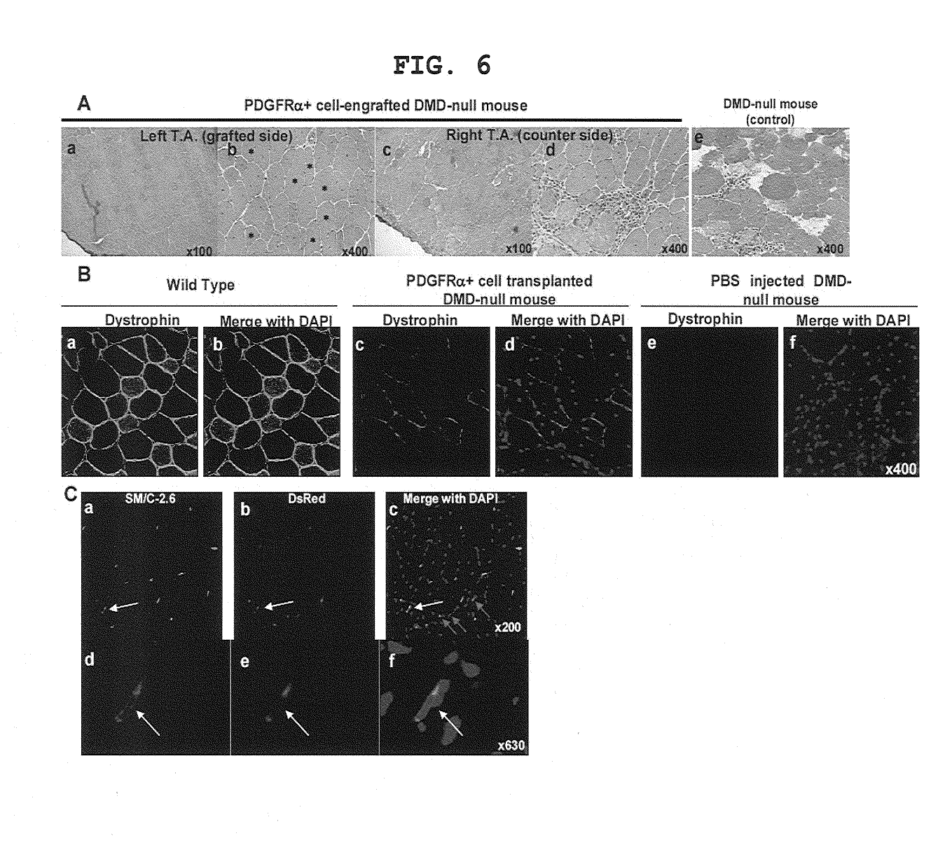

On day 6 of differentiation, PDGFRα-positive cells derived from iPS cells were separated by FACS, and transplanted to the left tibialis anterior muscle (T.A.) of 8-week old DMD-null mice by intramuscular injection. Four weeks later, the to tissue was analyzed by Hematoxylin-Eosin staining (FIG. 6A). In control DMD-null mice receiving no cells transplanted, the skeletal muscle interstitium was infiltrated by a very large number of inflammatory cells, with nuclei localized centrally, confirming that the skeletal muscles were amid regeneration (e). Meanwhile, in the T.A. receiving skeletal muscle progenitor cells derived from iPS cells transplanted by intramuscular injection, almost no sign of interstitial inflammation was observed (a). Although some skeletal muscle nuclei were localized centrally, many muscle fibers were found to have their nuclei shifted to the peripheries, suggesting completion of regeneration (b, marked with *). However, when the right T.A. (on the opposite side) free of transplantation of skeletal muscle progenitor cells derived from iPS cells was evaluated, severe interstitial infiltration of inflamed cells was evident (c), with nuclei localized centrally in the muscle fibers in almost all cases (d). These results confirmed that in the muscular dystrophy model mice, transplantation of skeletal muscle progenitor cells derived from iPS cells to skeletal muscles suppresses inflammation in the intramuscular injection site, which in turn leads to completion of muscle regeneration.

Subsequently, to determine whether the above-described histological changes are due to the expression of dystrophin, fluorescent immunostaining of dystrophin was performed (FIG. 6B). As expected in control DMD-null mice, no expression of dystrophin (visualized in green) was observed (e, f). However, in wild-type mouse positive controls, dystrophin is expressed in a mesh-like pattern around the peripheries of muscle fibers (a, b). In the T.A. receiving skeletal muscle progenitor cells derived from iPS cells transplanted thereto, dystrophin was expressed in a mesh-like and spot-like pattern around the peripheries of muscle fibers, though weaker than in the wild type (c, d). These results showed that skeletal muscle progenitor cells derived from iPS cells were engrafted as cells that produce muscle fiber dystrophin when transplanted to dystrophin-deficient muscular dystrophy model mice, resulting in the expression of dystrophin in the muscle fibers, whereby muscle fiber collapse is suppressed, the inflammation in the skeletal muscles is cured, and completion of the tissue repair and over-regeneration state is promoted.

To determine whether this tissue repair in the muscular dystrophy model mice is really by the action of the transplanted skeletal muscle progenitor cells derived from iPS cells, fluorescent immunostaining, including DsRed, was performed (FIG. 6C). As a result, in the T.A. receiving skeletal muscle progenitor cells derived from iPS cells transplanted thereto, DsRed-positive cells were observed mainly in the interstitium (FIG. 6C, c, red arrow), although DsRed-positive cells were also observed in the peripheral muscle fiber (FIG. 6C, b, e, white arrow), and SM/C-2.6 which is a marker of satellite cells was also expressed in the same site. Therefore, it was demonstrated that the skeletal muscle progenitor cells derived from iPS cells differentiated into satellite cells in the body (FIG. 6C, a, c, d, f, white arrow).

### Example 7

### Induction of In Vitro Differentiation of Skeletal Muscle Progenitor Cell Derived from iPS Cells

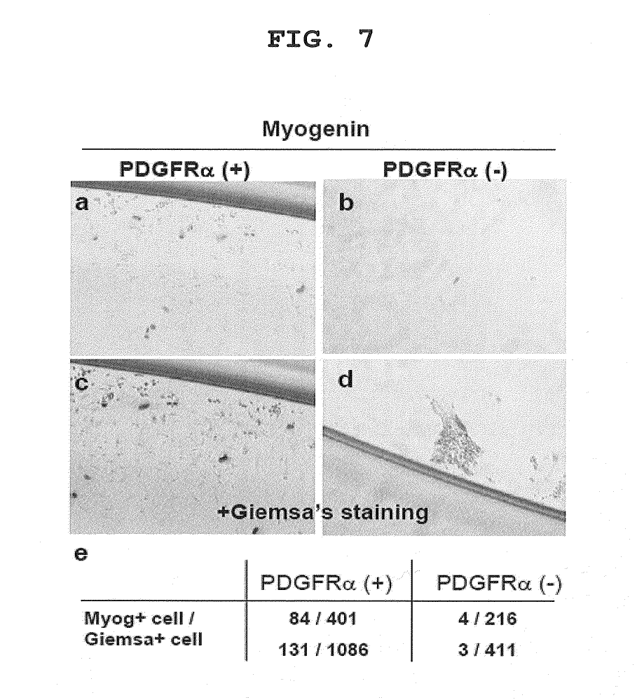

The two fractions (PDGFRα-positive fraction, PDGFRα-negative fraction) separated in Example 4 were further subjected to an experiment of differentiation of mature skeletal muscle in a test tube. Differentiation was induced according to the method described in “Reagents and methods”, 6. The results are shown in FIG. 7(a-e). While many Myogenin-positive cells, which are markers of mature skeletal muscle, differentiated from the PDGFRα-positive fraction (a, stained in brown), they were scarcely found in the PDGFRα-negative fraction (b). After Myogenin staining, the nucleus was co-stained by Giemsa staining. As a result, the Myogenin-positive part was identical with the nucleus in the PDGFRα-positive fraction, which certainly establishes that they were signals in the nucleus (c). It was also found that the Myogenin-negative nucleus was also present and not all cells had differentiated into the skeletal muscle. On the other hand, in the PDGFRα-negative fraction, most nuclei were Myogenin-negative (d). The proportion of the Myogenin-positive nuclei to the total number of nuclei on the culture dish is shown in the Table (e). In the PDGFRα-positive fraction, about 12-20% was differentiated into skeletal muscle, whereas in the PDGFRα-negative fraction, the appearance rate was extremely low and less than 2%. This demonstrated that the cell population having characters of skeletal muscle progenitor cells are for the most part contained in the PDGFRα-positive fraction.

### Example 8

### Study of Increase in Appearance Efficiency of Skeletal Muscle Progenitor Cell Derived from Ips Cells

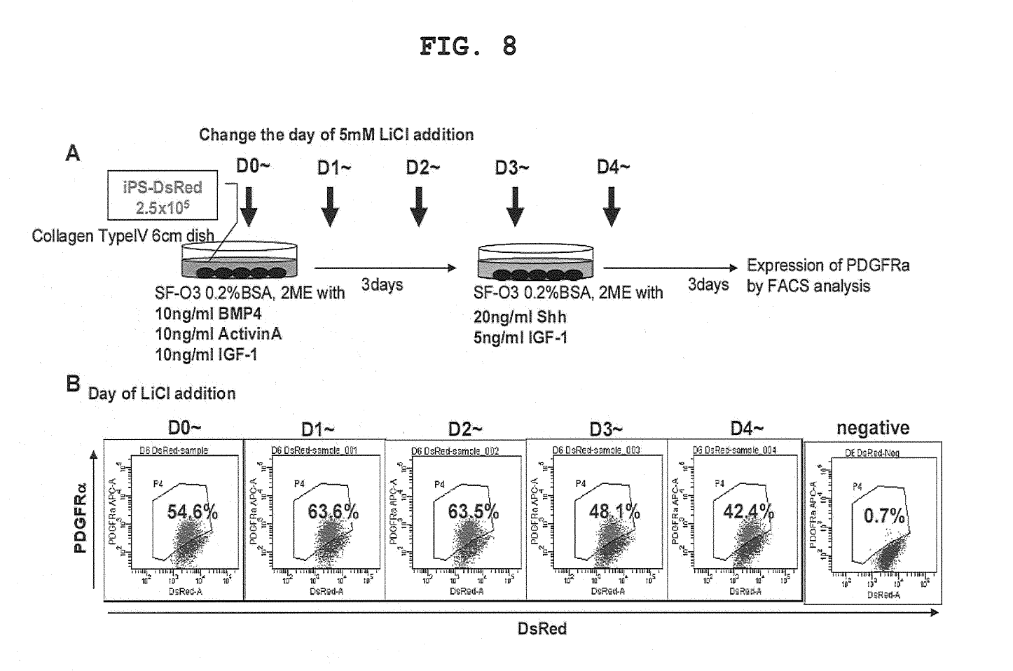

A method of increasing the appearance efficiency of skeletal muscle progenitor cells derived from iPS cells was studied. Specifically, LiCl, which was added on day 3 from the start of the differentiation induction in the previous Examples, was added from a different day. The protocol is shown in FIG. 8A. Samples with addition of LiCl from day 0, 1, 2, 3 or 4 from the start of the differentiation induction were analyzed for the appearance efficiency of the PDGFRα-positive fraction by FACS analysis. As a result, not less than 60% of the induction efficiency could be reproduced by continuous addition of LiCl from day 1 or day 2 from the start of the differentiation induction (FIG. 8B). This method could also be reproduced with other iPS cell clone (iPS-Ng-20D-17).

While the present invention has been described with emphasis on preferred embodiments, it is obvious to those skilled in the art that the preferred embodiments can be modified. The present invention intends that the present invention can be embodied by methods other than those described in detail in the present specification. Accordingly, the present invention encompasses all modifications encompassed in the gist and scope of the appended “CLAIMS.”

In addition, the contents disclosed in any publication cited herein, including patents and patent applications, are hereby incorporated in their entireties by reference, to the extent that they have been disclosed herein.

This application is based on U.S. provisional patent application No. 61/270,479 filed on Jul. 9, 2009, the contents of which are hereby incorporated by reference.

1.-6. (canceled)

7. A method of producing a skeletal muscle progenitor cell from a PDGFRα-positive mesodermal cell, wherein the mesodermal cell is cultured under serum-free conditions and in the presence of a Wnt signal inducer.

8. The method according to claim 7, wherein the skeletal muscle progenitor cell is Myf5-positive and MyoD-positive.

9. The method according to claim 7, wherein the Wnt signal inducer comprises at least one selected from among LiCl, Wnt1, Wnt3a and Wnt7a.

10. The method according to claim 7, wherein the mesodermal cell is cultured in the presence of further Shh and/or IGF-1.

11. The method according to claim 7, wherein the mesodermal cell has been obtained by culturing a pluripotent stem cell in the presence of Activin A.

12. A method of producing a skeletal muscle progenitor cell from a pluripotent stem cell, wherein the following steps 1) and 2) are followed under serum-free conditions:
1) the step of culturing a pluripotent stem cell in the presence of Activin A,
2) the step of culturing the cell obtained in the foregoing step 1) in the presence of a Wnt signal inducer.

13. The method according to claim 12, wherein the skeletal muscle progenitor cell is Myf5-positive and MyoD-positive.

14. The method according to claim 12, wherein the Wnt signal inducer comprises at least one selected from among LiCl, Wnt1, Wnt3a and Wnt7a.

15. The method according to claim 12, wherein the pluripotent stem cell is cultured in the presence of further BMP and/or IGF-1 in the foregoing step 1).

16. The method according to claim 15, wherein the BMP comprises at least one selected from among BMP2, BMP4 and BMP7.

17. The method according to claim 12, wherein the cell obtained in the foregoing step 1) is cultured in the presence of further Shh and/or IGF-1 in the foregoing step 2).

18. The method according to claim 12, wherein the following steps 1) and 2) are followed:
1) the step of culturing a pluripotent stem cell in the presence of Activin A, BMP4 and IGF-1,
2) the step of culturing the cell obtained in the foregoing step 1) in the presence of LiCl, Shh and IGF-1.

19. The method according to claim 12, wherein the foregoing steps 1) and 2) are followed by the step 3):
3) the step of selecting a PDGFRα-positive cell from among the cells obtained in the foregoing step 2).

20. The method according to claim 12, wherein the pluripotent stem cell is an iPS cell or ES cell.

21. A reagent kit for inducing differentiation from a pluripotent stem cell to a PDGFRα-positive mesodermal cell, wherein the kit comprises Activin A, BMP4 and IGF-1.

22. A reagent kit for inducing differentiation from a PDGFRα-positive mesodermal cell to a skeletal muscle progenitor cell, wherein the kit comprises LiCl, Shh and IGF-1.

23. The kit according to claim 21, wherein the pluripotent stem cell is an iPS cell or ES cell.

24.-31. (canceled)
